(19) United States
(12) Patent Application Publication
(10) Pub. No.: US 2023/0358758 A1
(43) Pub. Date: Nov. 9, 2023

(54) Assay for the detection of the Cys-like protease (Mpro) of SARS-CoV-2

(71) Applicant: CONSEJO SUPERIOR DE INVESTIGACIONES CIENTIFICAS (CSIC), Madrid (ES)

(72) Inventors: Hugh Thomson REYBURN, Madrid (ES); María Del Mar VALÉS GÓMEZ, Madrid (ES); José Miguel RODRÍGUEZ FRADE, Madrid (ES); José María CASASNOVAS SUELVES, Madrid (ES)

(73) Assignee: CONSEJO SUPERIOR DE INVESTIGACIONES CIENTIFICAS (CSIC), Madrid (ES)

(21) Appl. No.: 18/008,938

(22) PCT Filed: Jun. 8, 2021
(86) PCT No.: PCT/EP2021/065361

§ 371 Date: Dec. 7, 2022

(30) Foreign Application Priority Data
Jun. 8, 2020 (EP) 20382495.8

(51) Int. Cl. G01N 33/68; C07K 14/165

(52) U.S. Cl. CPC G01N 33/6854; C07K 14/165; G01N 2333/165

(57) ABSTRACT

In the present invention, we herein generally provide an in vitro method for detecting in at least one biological sample an antibody that binds to at least one epitope of the SARS-CoV-2 virus, comprising contacting said at least one biological sample with at least one isolated SARS-CoV-2 M^{pro} protein, or at least one fragment of said isolated SARS-CoV-2 M^{pro} protein comprising at least one epitope of the SARS-CoV-2 virus, and detecting the formation of an antigen-antibody complex between said virus protein or said fragment and an antibody present in said biological sample.

### TECHNICAL FIELD OF THE INVENTION

The present invention relates to recombinantly expressed proteins from the SARS-CoV-2, in particular to the main protease (M^{pro}, also known as 3CLPro) of SARS-CoV-2, as well as fragments thereof, and their use in the detection in a biological sample of antibodies that bind to at least one epitope of the SARS-CoV-2 virus.

### BACKGROUND OF THE INVENTION

In December 2019, a new coronavirus (CoV) emerged in China to cause an acute respiratory disease known as coronavirus disease 19 (COVID-19). The virus was identified to be a betacoronavirus related to severe acute respiratory syndrome coronavirus (SARS-CoV) and thus, was named SARS-CoV-2. In less than two decades, this virus is the third known coronavirus to cross the species barrier and cause severe respiratory infections in humans following SARS-CoV in 2003 and Middle East respiratory syndrome in 2012, yet with unprecedented spread compared to the earlier two. Due to the rapid rise in number of cases and uncontrolled and vast worldwide spread, the WHO has declared SARS-CoV-2 a pandemic. As of Mar. 14, 2020, the virus had infected over 130,000 individuals in 122 countries, 3.7% of which had a fatal outcome. The rapid identification of the aetiology and the sharing of the genetic sequence of the virus, followed by international collaborative efforts initiated due to the emergence of SARS-CoV-2 have led to the rapid availability of real-time PCR diagnostic assays that support the case ascertainment and tracking of the outbreak. The availability of these has helped in patient detection and efforts to contain the virus. However, specific and validated serologic assays are still lacking at the moment and are urgently needed to understand the epidemiology of SARS-CoV-2.

Validated serologic assays are crucial for patient contact tracing, identifying the viral reservoir hosts and for epidemiological studies. Epidemiological studies are urgently needed to help uncover the burden of disease, in particular, the rate of asymptomatic infections, and to get better estimates on morbidity and mortality. Additionally, these epidemiological studies can help reveal the extent of virus spread in households, communities and specific settings; which could help guide control measures. Serological assays are also needed for evaluation of the results of vaccine trials and development of therapeutic antibodies. Among the four coronavirus structural proteins, the spike (S) and the nucleocapsid (N) are the main immunogens (Meyer B, Drosten C, Muller M A. Serological assays for emerging coronaviruses: challenges and pitfalls. Virus research. 2014 Dec. 19; 194:175-83). The development of serological assays for the detection of virus neutralizing antibodies and antibodies to the nucleocapsid (NP) protein and various spike (S) domains including the S1 subunit, and receptor binding domain (RBD) of SARS-CoV-2 in ELISA format have been described.

In the present invention we provide for further viral protein immunogens useful for such assays, that are not structural and are not incorporated into the infectious viral particle.

### BRIEF DESCRIPTION OF THE FIGURES

FIG. 1. SARS-CoV-2 protein purification. (A) Schematic representation of proteins expressed from the plasmid constructs. Cys-like protease (3CLpro, Mpro), nucleocapsid (NP) and mammalian (mRBD) (B) SDS-PAGE. After expression in the different systems, proteins were purified and fractions from gel filtration chromatography were run in SDS-PAGE.

FIG. 2. Detection of SARS-CoV-2 Mpro-specific antibodies by ELISA. (A) Sera titration on Mpro. Plates were coated with SARS-CoV-2 Mpro and sera dilutions (1/50 to 1/1600) were tested. Detection was performed using anti-human F(ab)2′ antibody. (B) Isotype recognition. Plates coated with SARS-CoV-2 Mpro, nucleoprotein (NP) or RBD were detected with antibodies directed against human Ig of the three different subclasses: IgG, IgA, IgM. Black symbols correspond to covid-19 patients and grey symbols to donor samples collected pre-covid-19.

FIG. 3: Comparative ELISA tests. Plates coated with 0.5 ug/ml SARS-CoV-2 Mpro and nucleoprotein (NP) were used to perform the ELISA test on 36 COVID-19 positive patients (purple) and 33 negative controls. Detection was done using antibodies directed against human Ig of the three different subclasses: IgG, IgA, IgM. A) ELISA data of serum samples. B) ROC curves for the protease and NP assays of section A).

FIG. 4. Coating titration for detection of SARS-CoV-2-specific antibodies by ELISA. Plates were coated with SARS-CoV-2 M^{pro} and nucleoprotein (NP) and sera dilutions (1/50) to 1/1600) were tested. Detection was performed using antibodies directed against human Ig of the three different subclasses: IgG, IgA, IgM. Black symbols correspond to covid-19 patients and grey symbols to donors pre-covid-19.

FIG. 5: Comparison of Mpro and other viral antigens seroreactivity. Plates coated with either 0.5 ug/m SARS-CoV-2 Mpro or nucleoprotein (NP) or 1 μg/ml mRBD. serum samples diluted 1/100 were tested in ELISA assay and developed using anti-human IgG antibody. The two COVID19′ sera, negative sera and control wells are indicated.

FIG. 6. To explore the similarity between the Cys-like proteases of different coronaviruses, 3CLpro (Mpro) from SARS-CoV-2, HCovNL63 and HCov229E were aligned. The degree of similarity was around 40%.

FIG. 7. Background levels and negative controls. A. Background in plates with no viral protein coating. Plates were coated with 1 μg/ml of SARS-CoV-2 Mpro or iRBD and different dilutions of patient sera, as indicated, and detected with anti-human F(ab)2′ antibody (left and middle panels). Casein control corresponds to wells coated with the blocking solution, containing casein (right). These wells were incubated with the same sera and developed with anti-human F(ab)2′ antibody to check the background corresponding to individual sera. B. SARS-CoV-2 negative controls. 24 sera collected before 2020 (Pre-COVID-19) were tested in plates coated with 1 μg/ml of SARS-CoV-2 Mpro or NP. Sera were added at a 1/50-1/900 dilution. Detection was performed using antibodies directed against human IgG or IgM. Data from the 1/50 dilution are shown for IgM and 1/200 for IgG. Serum number 0850 corresponds to a positive control serum

FIG. 8. Comparison of sera from 33 pre-COVID-19 vs 36 COVID-19 patients. Plates coated with either 0.5 or 1 μg/ml (as indicated) SARS-CoV-2 M^{pro}, NP or RBD were used to perform ELISA tests on 36 COVID-19 positive and 33 negative control sera (obtained before the pandemic outbreak, PRE-COVID-19). Detection was done using antibodies directed against human immunoglobulin of the three different subclasses: IgG, IgA, IgM. Sera dilutions from 1/50-1/3200 were carried out. Data were normalised for each antigen using the signal obtained against a pool of positive sera. Box and whisker plots of all the sera tested at the 1/200 dilution for IgG and 1/50 for IgA and IgM. Statistical significance was analysed in Mann-Whitney tests. **** means p<0.0001.

FIG. 9. Assessment, through Receiver Operating Characteristic (ROC) analysis, of different isotype responses against three SARS-CoV-2 proteins as COVID-19 classifiers. Graphic representation of the relationship between sensitivity and specificity from analysis of 69 donors. The area under the curve (AUC) calculated for each antigen and immunoglobulin pair (see Statistical section of Material and Methods) is indicated.

FIG. 10. Comparisons between different sera dilutions for RBD, Mpro and NP. Plates coated with SARS-CoV-2 Mpro, NP or RBD were used to perform ELISA tests on 36 COVID-19 positive and 33 negative control sera. Detection was done using antibodies directed against human immunoglobulin of the three different subclasses: dilutions 1/50-1/3200 were used for IgG; dilutions 1/50-1/1350 were used for IgA and IgM. Graphs represent data of the ODs obtained for each antigen and each donor, after normalising the signal against a pool of positive sera. A. Mpro. B. RBD. C. NP.

FIG. 11. A. Correlations of humoral response against different SARS-CoV-2 antigens by isotype. Data from FIG. 8 are shown as dot-plots and their fitted fractional polynomial prediction with 95% confidence interval (transparent grey shadow) estimated using the two-way command of Stata with the fpfitci option. B. Comparison of sera from mild, severe and critical patients. Patients were classified into three groups (mild n=13, severe n=17 and critical n=6) according to COVID-19 symptoms severity (see reference 13). Data normalised for each antigen using the signal obtained against a pool of positive sera obtained in FIG. 2, are depicted in box and whisker plots at the 1/200 dilution for IgG and 1/50 for IgA and IgM. Statistical significance was analysed by Cuzick's test.

FIG. 12. Comparison of immunoglobulin levels at month 1 and 4 after COVID-19 symptoms onset. Plates were coated with SARS-CoV-2 NP, Mpro or RBD. Sera from 14 patients collected at different time points (during the first month and four-months after the onset of COVID-19, as indicated) were tested at a 1/200 dilution for IgG and 1/50 for IgA and IgM detection. All data were normalized for each antigen using the signal obtained with a pool of positive sera. A. OD variation for each donor. The graph relates samples for each donor within an isotype for each protein. The statistical significance was tested using a Wilcoxon test for paired samples. B. Percentage of variation. The same data as in A were plotted to visualise the percentage of variation normalizing to the first sample.

FIG. 13. Comparison of saliva from 11 healthy donors and 12 COVID-19 seropositive individuals. Plates coated with either 0.5 μg/ml of SARS-CoV-2 Mpro and NP or 1 μg/ml of RBD and ELISA tests were carried out on saliva samples diluted 1/2 to 1/10. Detection was done using antibodies directed against human IgG, IgM or IgA. Data were normalised for each antigen using the signal obtained for the positive control histidine-tag. Mann-Whitney test was performed to compare the values obtained for each dilution in healthy donors and patients. ** p<0.01, **** p<0.0001.

FIG. 14. A basic bead-assisted multiantigen assay for antibody detection in COVID-19 human serum samples. A. Schematic representation of the method. Four different SARSCoV-2 His-tagged antigens (Mpro, NP, S and RBD) were covalently coupled to magnetic beads labeled with dyes showing different fluorescence intensity in the APC and PerCP channels. Equal amounts of the different bead populations were mixed in the same tube and incubated with dilutions of plasma from patients or healthy donors, as indicated. Antibodies bound to the antigen were developed with fluorophore-conjugated anti-human Ig and samples were analysed by flow cytometry. B. Gating and antibody detection strategy. Magnetic beads coupled with individual SARS-CoV-2 antigens were mixed in a single well and incubated with the indicated dilutions of plasma from a healthy donor and a COVID-19 patients. Subsequently, detection was performed in two separate tubes, one with PEconjugated anti-human IgG and the second tube containing PE-conjugated anti-human IgM+FITC-conjugated anti-human IgA. The FSC/SSC region corresponding to 6 μm beads was selected and individual populations of beads were visualized in a APC/PerCP dot plot (left). Antibody bound to each bead type was analyzed independently in histograms within each bead gate. The plots represent Mean Fluorescence Intensity (MFI) values from the analysis of IgG obtained for the individual bead regions of one patient, comparing with a negative control (pre-COVID-19). C. Sera titration in the multi-antigenic assay. Antibody MFI values obtained for 6 healthy donors (closed symbols) and 6 COVID-19 patients (open symbols) in a multiantigen assay including a range of serum dilutions, as described in B.

FIG. 15. Heat map representing antibody titers from multi-antigen COVID-19 assays. Sera from 15 healthy controls and 29 COVID-19 patients were incubated with four different SARS-CoV-2 antigens coated beads: S, RBD, NP, and Mpro (indicated at the bottom), detected with antibodies to identify IgG, IgA and IgM and analysed by flow cytometry using the multi-antigen assay described in FIG. 14. To summarize all the data, a heat map was built showing the intensity of the IgG/IgM/IgA antibodies detected in donor sera. Each column corresponds to one antigen and rows include five different dilutions (1/100, 1/200, 1/600, 1/1800, 1/5400) for each individual. The intensity of the color depicts the amount of antibody.

FIG. 16. Multi-antigen serological assay identifies COVID-19 patients with nearly 100% confidence. A. Receiver Operating Characteristic (ROC) curves of single-antigen ELISA and multi-antigen FACS assays. A random forest classifier was trained with one healthy and 2 COVID controls IgG values and used to predict the rest of the samples. The mean ROC curve after 15-fold cross-validation is shown for each condition. B. Heat map of patients with biased IgG response against one type of viral antigens. Although most COVID-19 patients respond by producing antibodies against the four antigens tested, 5 out of 29 donors responded preferentially to either S/RBD or NP/Mpro. Data from 6 patients and 1 healthy donor are shown for comparison. C. Principal components Analysis (PCA). A principal component analysis was run with data from IgG antibodies (dilutions 1/100, 1/200, 1/600, 1/1800), and the two first principal components were used to represent each patient. Triangles and circles represent pre-pandemic controls and COVID-19 patients, respectively. D. PCA loadings. Visual representation of the loadings of the two first principal components of the PCA. Each dilution of IgG titer against RBD, Spike, NP and Mpro (as indicated) is represented as a separate variable.

FIG. 17. ROC curves classifying COVID-19 patients as either severe or mild disease. A random forest was trained to discriminate between COVID-19 patients with either severe or mild disease, using either igG data alone or including data from other isotypes, and then used to predict unseen patients (1/7 of total samples). The mean ROC curve after 300 random repetitions is shown for each condition.

FIG. 18. Titration of the anti-His antibody binding to individual viral antigen-coated beads. A. Estimation of the antibody-binding capacity of Mpro beads. Magnetic beads coated with SARS-CoV-2 Mpro were incubated with the indicated amounts of anti-his-tag antibody, followed by antirabbit-PE and analysed by flow cytometry. The histogram shows the fluorescence intensity for each anti-His concentration. B. Estimation of the antibody-binding capacity of SARS-CoV-2 antigen-coated beads. The same was done in parallel with different magnetic beads, each one coated with one SARS-CoV-2 antigen: Spike, the Receptor Binding Domain (RBD) of the S, the nucleocapsid protein (NP) and 3CL main protease (Mpro). Three dilutions of anti-His antibody were used: 16 ng/ml, 80 ng/ml and 400 ng/ml. The plots represent the MFI values obtained for each condition tested.

FIG. 19. FIG. 1. SARS-CoV-2 antigens. Nucleocapsid (NP) (A) and Cys-like protease (3CLpro, Mpro) (B) proteins were expressed in E. coli and extracted from the soluble fraction of the bacterial pellet. After selection in HiTrap Ni2+ chelating columns, the eluted fractions were run in SDS-PAGE (top gels). Proteins were further purified by gel filtration using a Superdex 75 column and run in SDS-PAGE (bottom gels). The FPLC profile is shown on the right panels. mRBD (C). The 334-528 fragment of the Spike protein was produced in mammalian cells fused to an HA-tag, at the N-terminus and to the TIM-1 mucin domain followed by the Fc portion of human IgG, at the C-terminus, with two thrombin-recognition sites (asterisks) which allowed release of the mRBDm-Fc fragment after treatment with thrombin [(+T) SDS-PAGE right panel]. After protein A and size exclusion chromatography (Superdex 75) purification SDS-PAGE was run under reducing conditions. Proteins bound (B) and unbound (U) to the protein A column are shown. (D) SDS-PAGE of purified proteins under non reducing conditions. E. Comparison of RBD-specific antibodies. Plates were coated with SARS-CoV-2 RBD proteins produced in eukaryotic systems, either using insect or mammalian cells, and sera dilutions (1/100 to 1/1600) were tested. Detection was performed using anti-human IgG antibody. Black symbols correspond to COVID-19 patients and grey symbols to samples from donors pre-COVID-19.

### DESCRIPTION OF THE INVENTION

The identification of the link between a novel betacoronavirus strain, named Severe Acute Respiratory Syndrome-CoronaVirus 2 (SARS-CoV-2), and a fatal respiratory illness, COVID-19, formally recognised as a pandemic by the WHO has led to a rush by health systems all over the world to develop and implement testing for viral infection. The rapid cloning and sequencing of the viral genome permitted the development of PCR-based assays for the detection of viral nucleic acids that have become a key strategy for both clinical diagnosis and epidemiological monitoring studies. However, PCR testing is not 100% efficient, indeed, it has been reported that the overall positive rate of RNA testing can be as low as 30-50% in patients with COVID-19 (Liu et al. Positive rate of RT-PCR detection of SARS-CoV-2 infection in 4880 cases from one hospital in Wuhan, China, from January to February 2020. Clin Chim Acta. 2020; 505:172-5; Yu et al. Quantitative Detection and Viral Load Analysis of SARS-CoV-2 in Infected Patients. Clin Infect Dis. 2020; Wang et al. The genetic sequence, origin, and diagnosis of SARS-CoV-2. Eur J Clin Microbiol Infect Dis. 2020). Further, testing for viral RNA requires specialised infrastructure and highly trained operators and, importantly, cannot detect evidence of past infection, which will be crucial for epidemiological efforts to assess how many people have been infected. Assays to measure antibody responses and determine seroconversion, while not appropriate to detect acute infections, are however, valuable sources of complementary information. Serological assays allow quantitative study of the immune response(s) to SARS-CoV-2 and are also critical for determination of the prevalence of infection in any given area; a necessary variable to define the infection fatality rate and that is of considerable utility for guiding management of the epidemic. Finally, quantitative and qualitative assays of antibody responses can aid in the identification of factors that correlate with effective immunity to SARS-CoV-2, the duration of these immune responses and may also aid in the selection of donors from whom preparations of convalescent serum/plasma can be generated for therapeutic use.

Multiple antibody tests to detect exposure to SARS-CoV-2, are becoming available, however the majority of these assays have been optimised to detect immunoglobulin G (IgG) antibodies to either the Spike (S) protein or the nucleoprotein of the virus. These proteins are key elements of the viral particle and are expected, by analogy with other coronaviruses, to be highly immunogenic. However, the immunogenicity of other viral proteins, 28 are encoded in the viral genome, has been little explored.

Here we have studied the antibody response to the main viral protease (Mpro, or 3CLPro) elicited after viral infection. Like other betacoronaviruses, SARS-CoV-2 is a positive-sense RNA virus that expresses all of its proteins as a single polypeptide chain. Mpro carries out the critical role in viral replication of cleaving the 1ab polyprotein to yield the mature proteins. Since this activity is essential for the viral life cycle, Mpro structure and function has been studied intensively as specific inhibitors of this enzyme might act as potent anti-viral agents. We now report, for the first time, that individuals who have been infected with SARS-CoV-2 make high titre antibody responses to Mpro and that assay for seroreactivity to this protein sensitively and specifically discriminates between infected and non-infected individuals.

Thus, generally, the present invention provides for isolated and recombinantly expressed SARS-CoV-2 M^{pro} proteins, and fragments thereof, for the detection of SARS-CoV-2 specific antibodies in infected humans.

### Definitions

In the context of the present invention the 3C-like protease (3CLpro, also referred to as the main protease, M^{pro}) of SARS-CoV-2 is characterized by having residues 3264-3569 of the ORF1ab polyprotein of GenBank accession code MN908947.3. This amino acid sequence is also characterized herein as SEQ ID NO 1 (from hereinafter referred to as “SARS-CoV-2 M^{pro} protein”.

A “fragment” of the SARS-CoV-2 M^{pro} protein according to the present invention is a partial amino acid sequence of the SARS-CoV-2 M^{pro} protein or a functional equivalent of such a fragment. A fragment is shorter than the complete SARS-CoV-2 M^{pro} protein and is preferably between about, 15, 20 or 65 and about 305 amino acids long, more preferably between about 15, 20 or 65 and about 250 amino acids long, even more preferably between about 65 and about 200 amino acids long. A fragment of the SARS-CoV-2 M^{pro} protein also includes peptides having at least 15, 20 or 65 contiguous amino acid residues having at least about 70%, at least about 80%, at least about 90%, preferably at least about 95%, more preferably at least 98% sequence identity with at least about 15, 20 or 65 contiguous amino acid residues of SEQ ID No. 1 having about the same length as said peptides. Depending on the expression system chosen, the protein fragments may or may not be expressed in native glycosylated form.

A fragment that “corresponds substantially to” a fragment of the SARS-CoV-2 M^{pro} protein is a fragment that has substantially the same amino acid sequence and has substantially the same functionality as the specified fragment of the SARS-CoV-2 M^{pro} protein.

A fragment that has “substantially the same amino acid sequence” as a fragment of the SARS-CoV-2 M^{pro} protein typically has more than 90% amino acid identity with this fragment. Included in this definition are conservative amino acid substitutions.

“Epitope” as used herein refers to an antigenic determinant of a polypeptide. An epitope could comprise three amino acids in a spatial conformation which is unique to the epitope. Generally, an epitope consists of at least five such amino acids, and more usually consists of at least 8-10 such amino acids. Methods of determining the spatial conformation of such amino acids are known in the art.

“Antibodies” as used herein are polyclonal and/or monoclonal antibodies or fragments thereof, including recombinant antibody fragments, as well as immunologic binding equivalents thereof, which are capable of specifically binding to the SARS-CoV-2 M^{pro} protein and/or to fragments thereof. The term “antibody” is used to refer to either a homogeneous molecular entity or a mixture such as a serum product made up of a plurality of different molecular entities. Recombinant antibody fragments may, e.g., be derived from a monoclonal antibody or may be isolated from libraries constructed from an immunized non-human animal.

“Sensitivity” as used herein in the context of testing a biological sample is the percentile of the number of true positive M^{pro} of SARS-CoV-2 samples divided by the total of the number of true positive M^{pro} of SARS-CoV-2 samples plus the number of false negative M^{pro} of SARS-CoV-2 samples.

“Specificity” as used herein in the context of testing a biological sample is the percentile of the number of true negative M^{pro} of SARS-CoV-2 samples divided by the total of the number of true negative M^{pro} of SARS-CoV-2 samples plus the number of false positive samples.

“Detection rate” as used herein in the context of antibodies specific for the SARS-CoV-2 virus is the percentile of the number of M^{pro} of SARS-CoV-2 positive samples in which the antibody was detected divided by the total number of M^{pro} of SARS-CoV-2 positive samples tested. “Overall detection rate” as used herein refers to the virus detection obtained by detecting both IgM and IgG.

A “clinical sample” comprises biological samples from one or from a random mix of patients, including patients with and without SARS-CoV-2.

“Onset of symptoms” as used herein is the onset of fever and a cough.

The terms “sequence identity” or “percent identity” in the context of two or more polypeptides or proteins refers to two or more sequences or subsequences that are the same (“identical”) or have a specified percentage of amino acid residues that are identical (“percent identity”) when compared and aligned for maximum correspondence with a second molecule, as measured using a sequence comparison algorithm (e.g., by a BLAST alignment, or any other algorithm known to persons of skill), or alternatively, by visual inspection.

A protein or peptide of the present invention has substantial identity with another if, optimally aligned, there is an amino acid sequence identity of at least about 60% identity with a naturally-occurring protein or with a peptide derived therefrom, usually at least about 70% identity, more usually at least about 80% identity, preferably at least about 90% identity, and more preferably at least about 95% identity, and most preferably at least about 98% identity. Identity means the degree of sequence relatedness between two polypeptide or two polynucleotides sequences as determined by the identity of the match between two strings of such sequences, such as the full and complete sequence. Identity can be readily calculated. While there exist a number of methods to measure identity between polypeptide sequences, the term “identity” is well known to skilled artisans.

### DESCRIPTION

In a first aspect of the present invention, we herein provide an in vitro method for detecting in at least one biological sample an antibody that binds to at least one epitope of the SARS-CoV-2 virus, comprising:
a. contacting said at least one biological sample with at least one isolated SARS-CoV-2 M^{pro} protein, or at least one fragment of said isolated SARS-CoV-2 M^{pro} protein comprising at least one epitope of the SARS-CoV-2 virus, and
b. detecting the formation of an antigen-antibody complex between said virus protein or said fragment and an antibody present in said biological sample.

It is herein noted, that to produce high amounts of the at least one isolated SARS-CoV-2 M^{pro} protein or at least one fragment of said isolated SARS-CoV-2 M^{pro} protein comprising at least one epitope of the SARS-CoV-2 virus thereof, the DNA fragments from genomic RNA can be produced by RT-PCR. The appropriate PCR primers can include restriction enzyme cleavage sites. After purification, the PCR products can be digested with the suitable restriction enzymes and cloned into suitable expression vectors, preferably, under the control of a strong promotor. The vectors then can be transformed into an appropriate host cell. Positive clones can be identified by PCR screening and further confirmed by enzymatic cut and sequence analysis. The so produced proteins/fragments then can be tested for their suitability as antigens for the method of the first aspect.

In a preferred embodiment of the first aspect, said at least one isolated SARS-CoV-2 M^{pro} protein is the protein of SEQ ID NO 1 or a variant of SEQ ID NO 1 having at least 80%, 85%, 90% or 95% sequence identity to SEQ ID NO 1, e.g., 95%, 96%, 97%, 98%, or 99%, sequence identity to SEQ ID NO 1.

In another preferred embodiment of the first aspect, said at least one fragment of said isolated SARS-CoV-2 M^{pro} protein comprising at least one epitope of the SARS-CoV-2 virus, comprises at least 15, 20 or 65 contiguous amino acid residues having at least about 70%, at least about 80%, at least about 90%, preferably at least about 95%, more preferably at least 98% sequence identity with at least about 15, 20 or 65 contiguous amino acid residues of SEQ ID No. 1. Preferably, said at least one fragment of said isolated SARS-CoV-2 M^{pro} protein has a sensitivity of more than about 70%, at least about 80%, at least about 90%, preferably at least about 95%, more preferably at least 98% or 99%. Preferably, said at least one fragment of said isolated SARS-CoV-2 M^{pro} protein has a specificity of more than about 70%, at least about 80%, at least about 90%, preferably at least about 95%, more preferably at least 98% or 99%. More preferably, said at least one fragment of said isolated SARS-CoV-2 M^{pro} protein has a detection rate or an overall detection rate of more than about 65%, at least about 70%, at least about 80%, at least about 90%, preferably at least about 95%, more preferably at least 98% or 99%.

In still another preferred embodiment of the first aspect or of any of its preferred embodiments, said method is adapted to detect IgG, IgM and/or IgA. Preferably, said method is adapted to detect IgG. In another preferred embodiment said method is adapted to detect IgG at a dilution of a fluid biological sample, preferably serum, of about 1:100, about 1:800, about 1:900, about 1:1000, about 1:1100 up to about 1:1200, 1:800 or 1:3200. In another preferred embodiment, the method according to the present invention is able to detect IgM at a dilution of a fluid biological sample, preferably serum, of about 1:50, about 1:100, about 1:200 up to about 1:400, or 1:1000.

In another preferred embodiment of the first aspect or of any of its preferred embodiments, said at least one isolated SARS-CoV-2 M^{pro} protein or fragment thereof is a recombinant expression product.

In still another preferred embodiment of the first aspect or of any of its preferred embodiments, appropriate biological samples to practice the method include, but are not limited to, mouth gargles, any biological fluids, virus isolates, tissue sections, wild and laboratory animal samples. Preferably, said biological sample is a blood, plasma or serum sample isolated from a subject, preferably a mammal, more preferably from a human being. Preferably, said biological sample is a serum or sera sample. In a preferred embodiment, the biological sample is mouth gargles, preferably saliva.

In still another preferred embodiment of the first aspect or of any of its preferred embodiments, said method is an in vitro diagnostic method for the detection of a subject having antibodies against the SARS-CoV-2 virus, wherein said subject is preferably a mammal, more preferably a human being, and wherein said subject is diagnosed as having antibodies against the SARS-CoV-2 virus if an antigen-antibody complex between said virus protein or said fragment and an antibody present in said biological sample is detected. In some embodiments, the mammal is selected from the group of human, mustelids or felines. In a further preferred embodiment, the mammal is selected from the group of cats, dogs, rats, cows, goats, sheep, horses, pigs, ferrets, human, rabbit, guinea pig, bovine and/or mouse. More preferably, the mammals are cats, human beings and/or ferrets.

In a preferred embodiment, said in vitro diagnostic method according to the present invention, will be able to additionally detect a wide array of stages of a SARS-CoV-2 infection. In still another preferred embodiment, said diagnostic method will be able to detect early stages of a SARS-CoV-2 infection. In another preferred embodiment, said diagnostic method will be able to detect early stages of infection by being able to detect IgM. In another preferred embodiment, said diagnostic method will be able to detect later stages of infection or a past infection by being able to detect IgG. In another preferred embodiment, said diagnostic method will be able to detect early stages of infection by being able to detect very low concentrations of antibodies. Accordingly, in a preferred embodiment the diagnostic method is adapted to detect antibodies against a SARS-CoV-2 virus less than about 50 days after the onset of symptoms, preferably less than about 40, less than about 30, less than about 25, less than about 20, less than about 15, less than about 12, less than about 10, less than about 9, less than about 8, less than about 7, less than about 6, less than about 5 less, or than about 4 days after the onset of symptoms.

In still another preferred embodiment of the first aspect or of any of its preferred embodiments, said method is an in vitro method for screening individuals having antibodies against the SARS-CoV-2 virus from those not having antibodies against the SARS-CoV-2 virus.

In still another preferred embodiment of the first aspect or of any of its preferred embodiments, the existence of antigen-antibody binding can be detected via methods well known in the art. In western blotting, one preferred method according to the present invention, fragments of a protein are transferred from the gel to a stable support such as a nitrocellulose membrane. The protein fragments can be reacted with sera from individuals suspected of having been infected with the SARS-CoV-2 virus. This step is followed by a washing step that will remove unbound antibody but retains antigen-antibody complexes. The antigen-antibody complexes then can be detected via anti-immunoglobulin antibodies which are labelled, e.g., with radioisotopes. Use of a western blot thus allows detection of the binding of sera of SARS-CoV-2 positive human to the M^{pro} of SARS-CoV-2 protein or a fragment thereof.

Other preferred detections methods include enzyme-linked immunosorbent assays (ELISA) and dot blotting. Both of these methods are relatively easy to use and are high throughput methods. ELISA, in particular, has achieved high acceptability with clinical personnel. ELISA, preferably based in chemiluminescent or colorimetric methods, is also highly sensitive. However, any other suitable method to detect antigen-antibody complexes such as, but not limited to, standardized radio immunoassays (RIA), lateral flow tests, also known as lateral flow immunochromatographic assays, or immunofluorescence assays (IFA), also can be used. Preferably, a specially preferred method is the ELISA assay, more preferably a chemiluminescent enzyme linked immunosorbent assay (ELISA).

In still another preferred embodiment of the first aspect or of any of its preferred embodiments, the method of detection is an ELISA assay by using the ELISA system as described later in the present specification.

In still another preferred embodiment of the first aspect or of any of its preferred embodiments, said biological sample is contacted with at least one or more further SARS-CoV-2 immunogens or fragments thereof, wherein preferably said immunogens are selected from the group consisting of nucleocapsid (N) proteins of SARS-CoV-2, and spike (S) domains including the S1 subunit, and/or receptor binding domain (RBD) of SARS-CoV-2.

In still another preferred embodiment of the first aspect or of any of its preferred embodiments, said biological sample is contacted with at least one or more further immunogens derived from at least one distinct isolated SARS protein.

A second aspect of the invention refers to an in vitro kit for detecting in a biological sample an antibody that binds to at least one epitope of the SARS-CoV-2 virus comprising:
a. at least one isolated SARS-CoV-2 M^{pro} protein, or at least one fragment of said isolated SARS-CoV-2 M^{pro} protein comprising at least one epitope of the SARS-CoV-2 virus, and
b. reagents for detecting the formation of antigen-antibody complex between said at least one isolated SARS-CoV-2 M^{pro} protein or a fragment thereof and at least one antibody present in a biological sample, wherein said at least one isolated protein or fragment thereof and said reagents are present in an amount sufficient to detect the formation of said antigen-antibody complex.

In a preferred embodiment of the second aspect of the invention, said at least one isolated SARS-CoV-2 M^{pro} protein is the protein of SEQ ID NO 1 or a variant of SEQ ID NO 1 having at least 80%, 85%, 90% or 95% sequence identity to SEQ ID NO 1, e.g., 95%, 96%, 97%, 98%, or 99%, sequence identity to SEQ ID NO 1.

In another preferred embodiment of the second aspect of the invention, said at least one fragment of said isolated SARS-CoV-2 M^{pro} protein comprising at least one epitope of the SARS-CoV-2 virus, comprises at least 15, 20 or 65 contiguous amino acid residues having at least about 70%, at least about 80%, at least about 90%, preferably at least about 95%, more preferably at least 98% sequence identity with at least about 15, 20 or 65 contiguous amino acid residues of SEQ ID No. 1.

In another preferred embodiment of the second aspect of the invention, said reagents are capable of detecting IgG, IgM and/or IgA. Preferably, said reagents are capable of detecting IgG. In another preferred embodiment said reagents are capable of detecting IgG at a dilution of about 1:100, about 1:800, about 1:900, about 1:1000, about 1:1100 up to about 1:1200. In another preferred embodiment, said reagents are capable of detecting IgM at a dilution of about 1:50, about 1:100, about 1:500 up to about 1:1000. In a particularly preferred embodiment, the kit is suitable for performing a radioimmunoassay (RIA), enzyme linked immunosorbent assay (ELISA) preferably a chemiluminescent or colorimetric enzyme linked immunosorbent assay (ELISA), immunofluorescence assay (IFA), dot blot, lateral flow test, also known as lateral flow immunochromatographic assay, or western blot. Preferably, the kit is an ELISA system.

In the context of the present invention, the ELISA (enzyme-linked immunosorbent assay) system is preferably understood as a plate-based assay technique designed for detecting and quantifying at least one isolated SARS-CoV-2 M^{pro} protein or fragment thereof. In this ELISA, the at least one isolated SARS-CoV-2 M^{pro} protein or fragment thereof must be immobilized to a solid surface and then exposed to the biological sample to form a complex. Detection is accomplished by any techniques well known in the art.

ELISAs, according to the present invention, are typically performed in 96-well (or 384-well) polystyrene plates, which will passively bind at least one isolated SARS-CoV-2 M^{pro} protein or fragments thereof. The binding and immobilization of reagents makes ELISAs simple to design and perform. Having the reactants of the ELISA immobilized to the microplate surface enables easy separation of bound from non-bound material during the assay. This ability to wash away non-specifically bound materials makes the ELISA a powerful tool for measuring specific analytes within a crude preparation.

In a preferred embodiment of the second aspect of the invention the ELISA system comprises at least one isolated SARS-CoV-2 M^{pro} protein or fragments thereof to coat or coating a solid surface, preferably microtiter plate wells, and optionally one or more of the following reagents: blocking reagents for unbound sites to prevent false positive results; anti-(species) IgG, IgM and/or IgA conjugated to a label, preferably an enzyme; and substrates that react with the label, preferably the enzyme, to indicate a positive reaction. In addition to the procedure reagents, additional reagents such as wash buffers, stop solutions and stabilizers can enhance the quality of the ELISA assay. When choosing individual reagents, or complete kits, it is helpful to know the sensitivity required and whether one is trying to detect an analyte or the antibody response to it.

In still another preferred embodiment of the second aspect of the invention, said at least one isolated SARS-CoV-2 M^{pro} protein or fragment thereof is a recombinant expression product.

In still another preferred embodiment of the second aspect of the invention, said at least one isolated SARS-CoV-2 M^{pro} protein is the protein of SEQ ID NO 1.

A third aspect of the invention refers to the use of kit of the second aspect of the invention or of any of its preferred embodiments, for implementing any of the methods disclosed in the first aspect of the invention.

The uses of the at least one isolated SARS-CoV-2 M^{pro} protein, or at least one fragment of said isolated SARS-CoV-2 M^{pro} protein comprising at least one epitope of the SARS-CoV-2 virus, according to the present invention that are described above are those which presently appear most attractive. However, the foregoing disclosures of embodiments of the invention and uses therefor have been given merely for purposes of illustration and not to limit the invention. Thus, the invention should be considered to include all embodiments falling within the scope of the claims following the Example section and any equivalents thereof.

The following examples are merely illustrative and should not be construed to limit in any way the invention as set forth in the claims which follow.

### EXAMPLES

The following sequences were used to carried out the examples and figures illustrated in the present description and figures:

Sequence listing

Peptide sequence SARS-COV-2 Mpro (SEQ ID NO 1):
SAVLQSGFRKMAFPSGKVEGCMVQVTCGTTTLNGLWLDDVVYCPRHVIC
TSEDMLNPNYEDLLIRKSNHNFLVQAGNVQLRVIGHSMQNCVLKLKVDT
ANPKTPKYKFVRIQPGQTFSVLACYNGSPSGVYQCAMRPNFTIKGSFLN
GSCGSVGFNIDYDCVSFCYMHHMELPTGVHAGTDLEGNFYGPFVDRQTA
QAAGTDTTITVNVLAWLYAAVINGDRWFLNRFTTTLNDFNLVAMKYNYE
PLTQDHVDILGPLSAQTGIAVLDMCASLKELLQNGMNGRTILGSALLED
EFTPFDVVRQCSGVTFQLEHHHHHH
Nucleotide sequence of SARS-COV-2 Mpro
(SEQ ID NO 2):
tcagctgttttgcagagtggttttagaaaaatggcattcccatctggta
aagttgagggttgtatggtacaagtaacttgtggtacaactacacttaa
cggtctttggcttgatgacgtagtttactgtccaagacatgtgatctgc
acctctgaagacatgcttaaccctaattatgaagatttactcattcgta
agtctaatcataatttcttggtacaggctggtaatgttcaactcagggt
tattggacattctatgcaaaattgtgtacttaagcttaaggttgataca
gccaatcctaagacacctaagtataagtttgttcgcattcaaccaggac
agactttttcagtgttagcttgttacaatggttcaccatctggtgttta
ccaatgtgctatgaggcccaatttcactattaagggttcattccttaat
ggttcatgtggtagtgttggttttaacatagattatgactgtgtctctt
tttgttacatgcaccatatggaattaccaactggagttcatgctggcac
agacttagaaggtaacttttatggaccttttgttgacaggcaaacagca
caagcagctggtacggacacaactattacagttaatgttttagcttggt
tgtacgctgctgttataaatggagacaggtggtttctcaatcgatttac
cacaactcttaatgactttaaccttgtggctatgaagtacaattatgaa
cctctaacacaagaccatgttgacatactaggacctctttctgctcaaa
ctggaattgccgttttagatatgtgtgcttcattaaaagaattactgca
aaatggtatgaatggacgtaccatattgggtagtgctttattagaagat
gaatttacaccttttgatgttgttagacaatgctcaggtgttactttcc
aactcgagcaccaccaccaccaccactga
Peptide sequence SARS-COV-2 nucleocapsid protein
(SEQ ID NO 3):
MASDNGPQNQRNAPRITFGGPSDSTGSNQNGERSGARSKQRRPQGLPNN
TASWFTALTQHGKEDLKFPRGQGVPINTNSSPDDQIGYYRRATRRIRGG
DGKMKDLSPRWYFYYLGTGPEAGLPYGANKDGIIWVATEGALNTPKDHI
GTRNPANNAAIVLQLPQGTTLPKGFYAEGSRGGSQASSRSSSRSRNSSR
NSTPGSSRGTSPARMAGNGGDAALALLLLDRLNQLESKMSGKGQQQQGQ
TVTKKSAAEASKKPRQKRTATKAYNVTQAFGRRGPEQTQGNFGDQELIR
QGTDYKHWPQIAQFAPSASAFFGMSRIGMEVTPSGTWLTYTGAIKLDDK
DPNFKDQVILLNKHIDAYKTFPPTEPVEHHHHHH
Nucleotide sequence of SARS-CoV-2 nucleocapsid
protein (SEQ ID NO 4):
atggcttctgataatggtccgcaaaatcagcgtaatgcaccccgcatta
cgtttggtggaccctcagattcaactggcagtaaccagaatggagaacg
cagtggggcgcgatcaaaacaacgtcggccccaaggtttacccaataat
actgcgtcttggttcaccgctctcactcaacatggcaaggaagacctta
aattccctcgaggacaaggcgttccaattaacaccaatagcagtccaga
tgaccaaattggctactaccgaagagctaccagacgaattcgtggtggt
gacggtaaaatgaaagatctcagtccaagatggtatttctactacctag
gaactgggccagaagctggacttccctatggtgctaacaaagacggcat
catatgggttgcaactgagggagccttgaatacaccaaaagatcacatt
ggcacccgcaatcctgctaacaatgctgcaatcgtgctacaacttcctc
aaggaacaacattgccaaaaggcttctacgcagaagggagcagaggcgg
cagtcaagcctcttctcgttcctcatcacgtagtcgcaacagttcaaga
aattcaactccaggcagcagtaggggaacttctcctgctagaatggctg
gcaatggcggtgatgctgctcttgctttgctgctgcttgacagattgaa
ccagcttgagagcaaaatgtctggtaaaggccaacaacaacaaggccaa
actgtcactaagaaatctgctgctgaggcttctaagaagcctcggcaaa
aacgtactgccactaaagcatacaatgtaacacaagctttcggcagacg
tggtccagaacaaacccaaggaaattttggggaccaggaactaatcaga
caaggaactgattacaaacattggccgcaaattgcacaatttgccccca
gcgcttcagcgttcttcggaatgtcgcgcattggcatggaagtcacacc
ttcgggaacgtggttgacctacacaggtgccatcaaattggatgacaaa
gatccaaatttcaaagatcaagtcattttgctgaataagcatattgacg
catacaaaacattcccaccaacagagcctgtcgagcaccaccaccacca
ccactga
Peptide sequence SARS-COV-2 RBD (receptor binding
domain) (SEQ ID NO 5):
gsrNLCPFGEVFNATRFASVYAWNRKRISNCVADYSVLYNSASFSTFKC
YGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCG
PKIvpr
Nucleotide sequence of SARS-COV-2 RBD
(SEQ ID NO 6):
ggatcccgcaacttgtgcccttttggtgaagtttttaacgccaccagat
ttgcatctgtttatgcttggaacaggaagagaatcagcaactgtgttgc
tgattattctgtcctatataattccgcatcattttccacttttaagtgt
tatggagtgtctcctactaaattaaatgatctctgctttactaatgtct
atgcagattcatttgtaattagaggtgatgaagtcagacaaatcgctcc
agggcaaactggaaagattgctgattataattataaattaccagatgat
tttacaggctgcgttatagcttggaattctaacaatcttgattctaagg
ttggtggtaattataattacctgtatagattgtttaggaagtctaatct
caaaccttttgagagagatatttcaactgaaatctatcaggccggtagc
acaccttgtaatggtgttgaaggttttaattgttactttcctttacaat
catatggtttccaacccactaatggtgttggttaccaaccatacagagt
agtagtactttcttttgaacttctacatgcaccagcaactgtttgtgga
cctaaacttgtccctcgc

### Example 1

### Materials and Methods

### Molecular Cloning of the SARS-CoV-2 Cys-Like Protease (3CLpro, Mpro) and Nucleocapsid Proteins (NP).

A gene encoding SARS-CoV-2 Mpro (ORF1ab polyprotein residues 3264-3569, GenBank code:MN908947.3) was amplified by PCR using the oligos 5′-gacccatggcttcagctgtttttcagagtggttt-3′ and 5′-gacctcgagttggaaagtaacacctgagcatt-3′, digested with NcoI and XhoI and ligated into the vector pET22b (Novagen) digested with the same restriction enzymes. The integrity of this construct was verified by sequencing at MWG Eurofins.

Oligonucleotides 5′-gatccatggcttctgataatggtccgcaaaatcagcgtaatgca-3′ and 5′-caggtcgacaggctctgttggtgggaatg-3′ were used to amplify the nucleocapsid protein (NP) of SARS-CoV-2. The amplification product was then digested with NcoI and SalI and ligated into the pET26b vector (Novagen) digested with NcoI and XhoI.

The integrity of all constructs was verified by sequencing at MWG Eurofins.

### Expression of the SARS-CoV-2 Cys-Like Protease (3CLpro, Mpro) and Nucleocapsid Proteins

SARS-CoV-2 Mpro protein was expressed by transforming this plasmid into the E. coli strain BL21 Star (DE3) pLysS (ThermoFisher). Transformed clones were pre-cultured overnight at room temperature in 50 mL 1×LB medium with ampicillin (150 μg/mL) and chloramphenicol (34 ug/ml). The overnight culture was then inoculated into 1 L of 1×LB medium (150 μg/mL ampicillin and 34 ug/ml chloramphenicol) and the culture was grown at 37° C. with agitation until the OD₆₀₀ reached 0.6 when Isopropyl-D-thiogalactoside (IPTG) was added to 1 mM to induce overexpression of the Mpro gene. The same protocol was followed to produce nucleocapsid protein except that kanamycin (150 ug/ml) was used instead of ampicillin for antibiotic-mediated selection.

After overnight culture at 22° C., bacteria were harvested by centrifugation at 9500×g, 4° C. for 15 min and the pellets were washed by resuspension in 150 mL TES buffer (20 mM Tris pH 8, 2 mM EDTA, 150 mM NaCl) and re-centrifugation. Washed pellets were either processed immediately or stored frozen for later use.

Fresh, or thawed, cell pellets were resuspended in ice-cold 50 mM NaH₂PO₄ buffer pH8, 500 mM NaCl, 10 mM imidazole (12399, Sigma Aldrich), 0.1% Sarkosyl, and 5% glycerol (pH 8.0). Lysozyme was then added (to 0.25 mg/ml) as were phenylmethylsulfonyl fluoride, Leupeptin and Pepstatin A (all to a final concentration of 1 mM) and DNase 1 (2 μg/ml). Bacteria were lysed by sonication (3 cycles of 30 seconds with 30 seconds rest on ice between pulses) and soluble proteins were separated by centrifugation of the lysed cells at 14,000 g at 4° C. for 45 minutes.

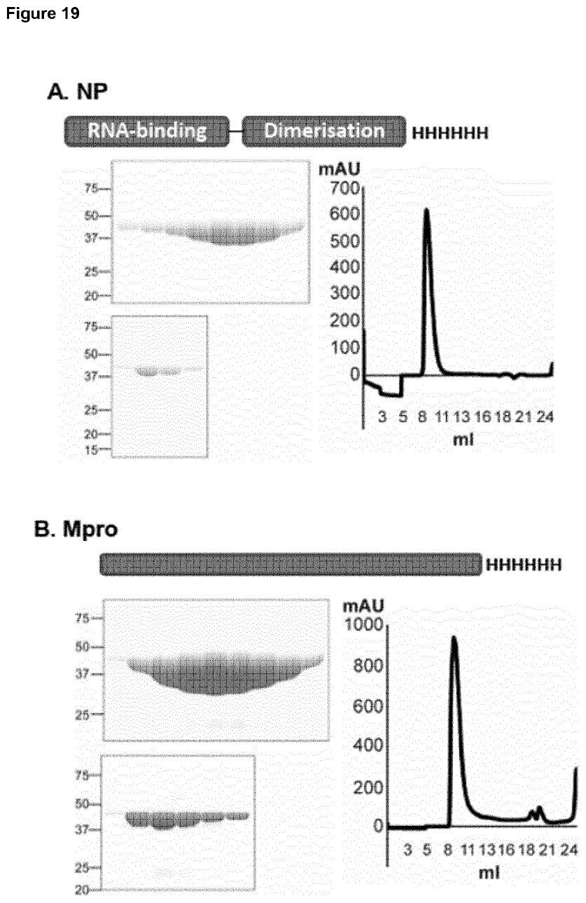

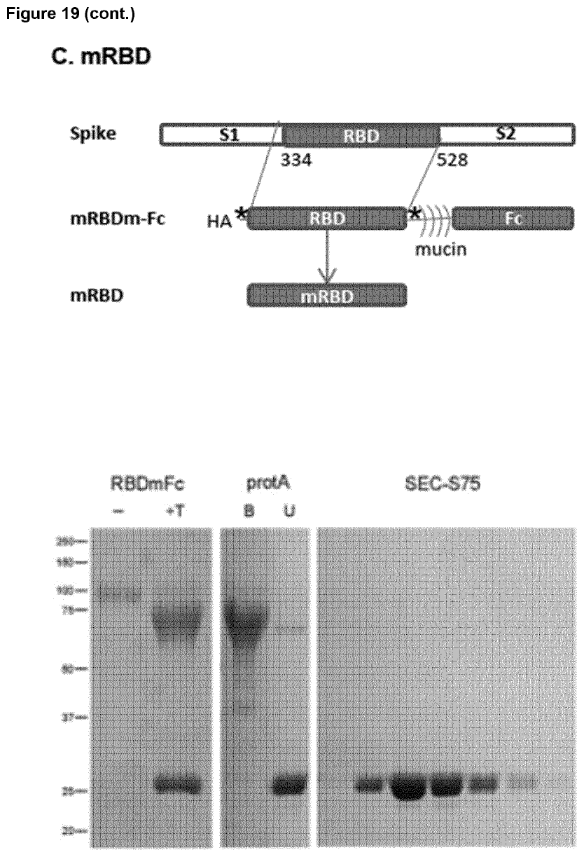

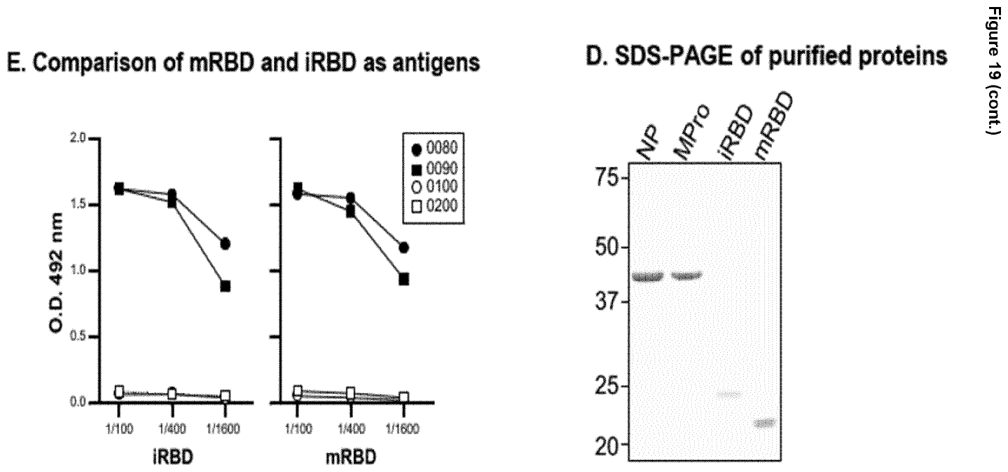

6-histidine tagged proteins were purified from the lysate using 5-ml HiTrap Ni2+ chelating columns (Agarose bead technologies). The bacterial supernatant was loaded on the column at a flow rate of 1 ml/min, followed by washing with 5 column volumes of 50 mM NaH₂PO₄ buffer, 500 mM NaCl, 10 mM imidazole and then 5 column volumes of 50 mM NaH₂PO₄ buffer, 500 mM NaCl, 25 mM imidazole. Recombinant proteins were eluted using a linear gradient of imidazole ranging from 25 mM to 250 mM over 5 column volumes (a representative SDS-PAGE analysis of the eluted fractions is shown in FIG. 19A). The proteins were then further purified by gel filtration using a 10/30 Superdex 75 Increase column (Cytiva) pre-equilibrated in 10 mM Tris, 2 mM EDTA, 300 mM NaCl, pH 7.5. The gel filtration analysis indicated that the SARS CoV 2 Mpro protein purified as a dimer.

Molecular cloning and production of SARS-CoV-2 Receptor Binding Domain protein in mammalian cells (mRBD).

The cDNA region coding for the Receptor Binding Domain (RBD) (residues 334-528) defined in the structure of the S protein (PDB ID 6VSB) was amplified for expression in mammalian cells. The fragment was cloned in frame with the IgK leader sequence, an HA-tag (YPYDVPDYA) and a thrombin recognition site (LVPRGS) at its 5′ end, and it was followed by a second thrombin site, the TIM-1 mucin domain and the human IgG1 Fc region at the 3′ end. The recombinant cDNA was cloned in a vector derived from the pEF-BOS (9) for transient expression in HEK293 cells, and in the pBJ5-GS vector for stable protein production in CHO cells following the glutamine synthetase system (Casasnovas J M, and Springer T A. Kinetics and thermodynamics of virus binding to receptor. Studies with rhinovirus, intercellular adhesion molecule-1 (ICAM-1), and surface plasmon resonance. The Journal of biological chemistry. 1995; 270(22):13216-24). The inclusion of the TIM-1 mucin domain enhanced protein expression.

Mammalian RBD (mRBD) fused to the mucin domain and the Fc region (mRBD-mucin-Fc) was initially purified from cell supernatants by affinity chromatography using an IgSelect column (GE Healthcare). The mucin-Fc portion and the HA-tag were released from the mRBD protein by overnight treatment with thrombin at RT. The mixture was run through a protein A column to remove the mucin-Fc protein and mRBD was further purified by size-exclusion chromatography with a Superdex 75 column in HBS buffer (25 mM HEPES and 150 mM NaCl, pH 7.5). The concentration of purified mRBD was determined by absorbance at 280 nm.

### ELISA for Detection of Antibodies to SARS-CoV-2

96-well Maxisorp Nunc-Immuno plates were coated with 100 μL/well of recombinant proteins diluted in borate buffered saline (BBS, 10 mM borate, 150 mM NaCl, pH 8.2); NP and the protease at 0.5 μg/ml, RBD at 1 μg/ml and incubated overnight at 4° C. Coating solutions were then aspirated, the ELISA plates were washed three times with 200 μl of PBS 0.05% Tween 20 (PBS-T) and then dried before blocking with PBS with 1% casein (Biorad) for 2 hours at room temperature. The plates were washed again with PBS-T and 100 μl of patient serum/plasma sample diluted in PBS-casein, 0.02% Tween-20, as indicated, was added and incubated for 2 hours at 37° C. The plates were washed again and 100 μL/well of the indicated detection antibody, all from Jackson Labs (AffiniPure Rabbit Anti-Human IgM, Fcp fragment specific. HRPO, AffiniPure Rabbit Anti-Human Serum IgA, a chain specific. HRPO, or AffiniPure Rabbit Anti-Human IgG, Fcγ fragment specific. HRPO) was added and incubated for 1 hour at room temperature. The plates were washed with PBS-T four times and incubated at room temperature in the dark with 100 μL/well of Substrate Solution (OPD, Sigma prepared according to the manufacturer's instructions) (typically for 3 minutes). 50 μL of stop solution (3M H₂SO₄) were then added to each well and the optical density (at 492 nm) of each well was determined using a microplate reader.

Negative controls included wells coated just with blocking buffer and serum samples collected from donors before 2019.

### Statistical Analysis

Graphics and statistical analysis was performed with Graph Pad Prism 8 Software (GraphPad Software, USA, www.graphpad.com) and Stata 14.0 for Windows (Stata Corp LP, College Station, TX, USA). Quantitative variables following a non-normal distribution were represented as median and interquartile range (IQR) and the Mann Whitney test was used to test for statistically significant differences. Variables with a normal distribution were described by mean±standard deviation (SD) and differences between groups were assessed with Student's t-test. Qualitative variables were described as counts and proportions and χ2 or Fisher's exact test was used for comparisons. Correlation between quantitative variables was analysed using the Pearson correlation test.

Severity of COVID-19 was established as previously described (Ibarrondo et al. 2020. Rapid decay of anti-SARS-CoV-1 antibodies in persons with mild covid-19. N Engl J Med). In this case, to determine differences in titres of antibodies between groups of severity the Cuzick's test, that assesses trends across ordered groups, was employed.

Since several variables might contribute to differences in ELISA titres, multivariable linear analysis using generalized linear models (glm command of Stata) in which the dependent variable were ELISA titres of each isotype against each protein. The first model included age, gender and time from symptoms onset, followed by backward stepwise approach removing all variables with a p value>0.15 to obtain the best model for each protein and isotype. Then, the variable of interest (severity, anosmia or IL-6 serum levels) was forced in the model.

To determine the capacity of the different ELISA to discriminate between pre-COVID-19 sera and those sera obtained from patients with SARS-CoV-2, as determined by positive PCR from nasopharyngeal exudates, ROC analysis was performed, using the roctab command of Stata 14.1@ (College Station, Texas). Each cut-off point was selected based on the best trade-off values between sensitivity, specificity and the percentage of patients correctly classified. ROC curves and area under curve (AUC) were also obtained.

### Patient Samples and Institutional Review Boards

This study used samples from the research project “Immune response dynamics as predictor of COVID-19 disease evolution. Implications for therapeutic decision-making” [PREDINMUN-COVID] approved by La Princesa Health Research Institute (IIS-IP) Research Ethics Committee (register #4070). Some experiments included patients from “Study of the lymphocytic response against SARS-COV-2, in different situations of host health and COVID-19 severity (InmunoCOVID)” approved by the Hospital La Paz Committee (HULP: PI-4101). All experiments were carried out following the ethical principles established in the Declaration of Helsinki. All included patients (or their representatives) were informed about the study and gave a written informed consent.

### Patient Selection

36 COVID-19 patients, diagnosed by PCR, were recruited for the study. 9 of them presented active infection by SARS-CoV2 at the moment of the study whereas the rest had no detectable levels of the virus. 10 patients required hospitalization, of which 6 were admitted to the ICU (Table 1). 33 serum samples from patients presenting a monoclonal gammopathy, allergic disease or rheumatoid arthritis, collected before June 2019 (PRE-COVID-19), were used as negative controls. All samples were stored frozen before use.

### Antibody Detection in Saliva Samples

12 donors with high antibody titres in serum were selected to measure specific IgG and IgA against SARS-CoV2 in saliva. For this purpose, new saliva samples were collected from these patients, and also from 11 healthy donors, aliquoted and immediately frozen. Prior to use, saliva samples were thawed, centrifuged at 400 g and diluted 1/2, 1/4 and 1/10 in 1×PBS with 1% casein (Bio-Rad) and 0.02% Tween-20 supplemented with Complete™ Protease Inhibitor Cocktail (Roche).

### Results

Production of Soluble Cys-Like Protease (3CLpro, Mpro) from SARS-CoV-2.

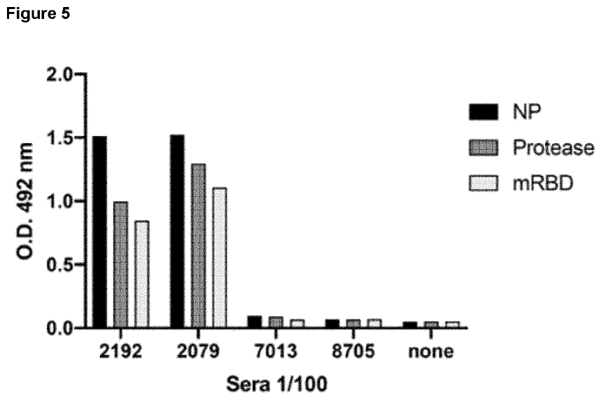

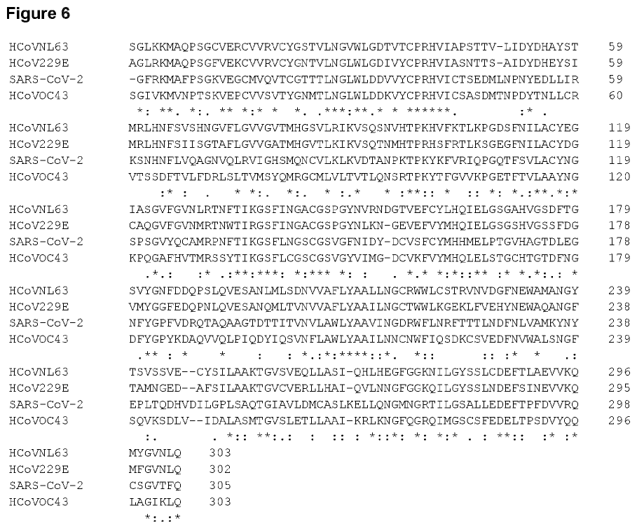

To explore the similarity between the Cys-like proteases of different coronaviruses, 3CLpro (Mpro) from SARS-CoV-2, HCovNL63 and HCov229E were aligned (FIG. 6). The degree of similarity was around 40%.

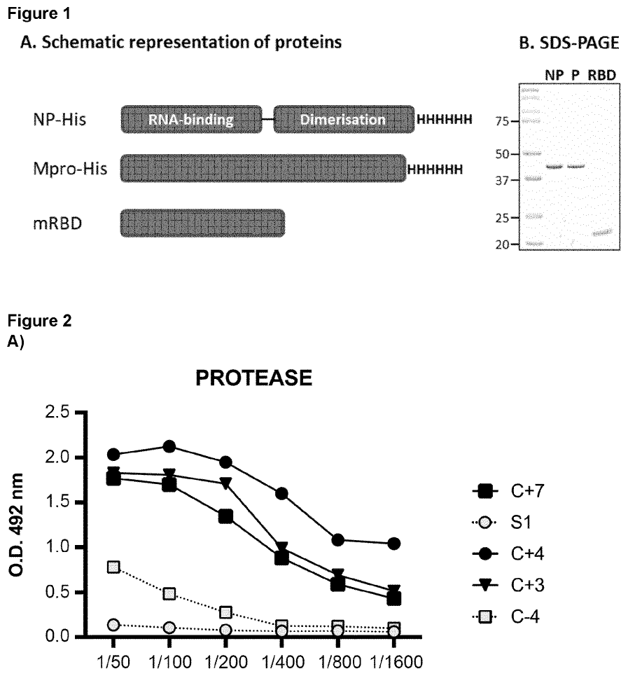

The nucleotide sequence (SEQ ID NO 2) from the SARS-CoV-2 Cys-like protease (also known as 3CLpro, Mpro) from the Wuhan-Hu-1 strain (GenBank accession number MN908947.3) was amplified and subcloned into the prokaryotic expression vector pET22b and the soluble protein was expressed in the E. coli strain BL21 Star (DE3) pLysS. After extraction of the soluble proteins from the bacterial lysates and selection of the His-tagged proteins in a Ni-NTA column, Mpro was purified by size exclusion yielding a protein with an apparent Mw of around 70 kDa, corresponding to a dimer. FIG. 1 shows schematic representations and SDS-PAGE data of the recombinant proteins.

Since the aim was to evaluate, for the first time, if coronavirus-infected individuals could generate an antibody response against Cys-like proteases, other SARS-CoV-2 proteins were also produced: NP (SEQ ID NO 4) was expressed in E coli, and RBD was expressed by transfection in mammalian cells (mRBD of SEQ ID NO 5). NP and protease had a Histidine tag and they were purified on Ni²⁺-NTA columns followed by size exclusion.

### High Titres of SARS-CoV-2 3CLpro-Specific Antibodies can be Detected in Covid-19 Positive Patient Sera but not in Negative Donors.

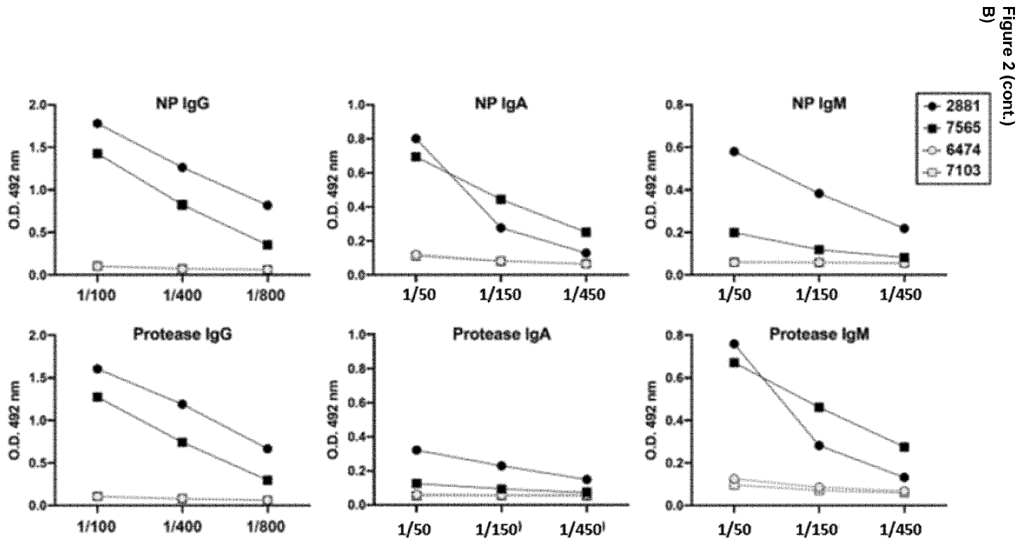

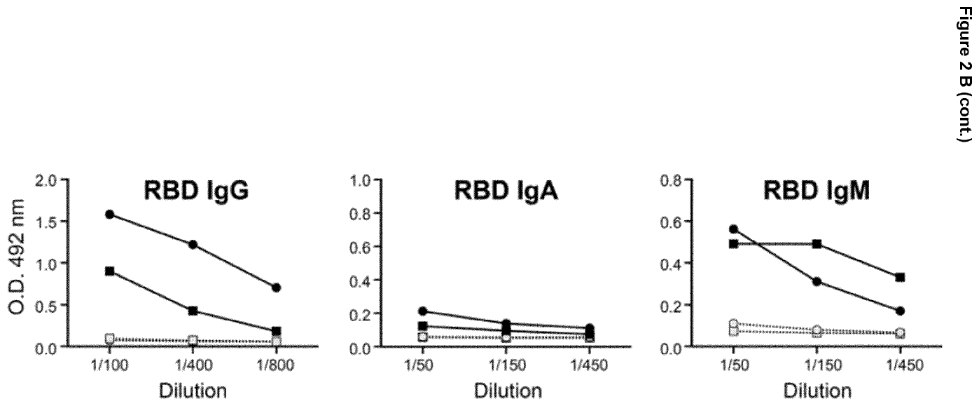

First experiments using covid-19 patient sera revealed the presence of Mpro antibodies at high titres. Antibody reactivity to the protease reached saturation at low concentrations and discriminated efficiently between individuals who had been infected with SARS-CoV-2 and those that did not have the disease (FIG. 2A). Further, it was possible to detect antibodies of the three isotypes, IgG, IgM and IgA. The reactivity of the different sera against the protease Mpro was comparable or in certain cases stronger to the reactivity against the other viral protein (FIG. 2B).

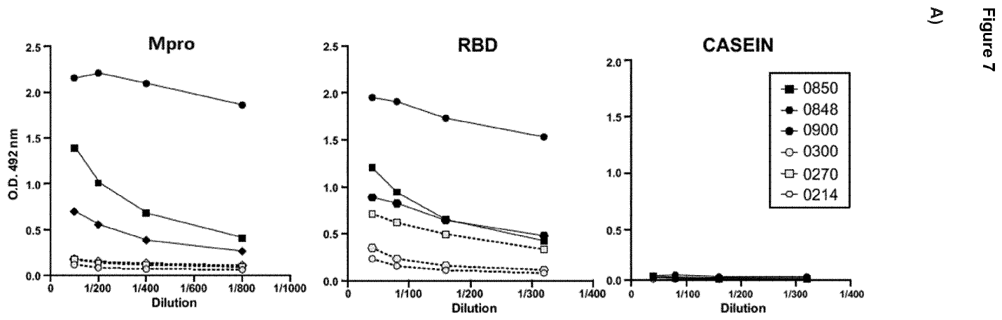

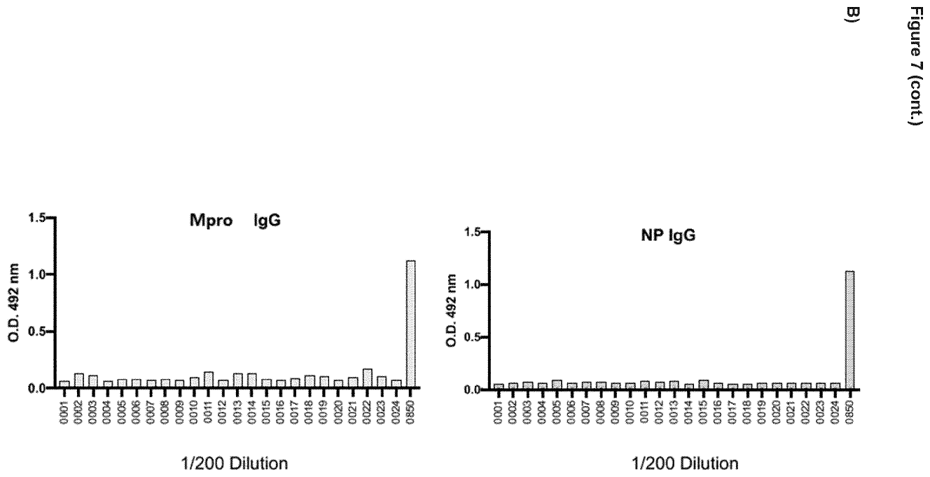

Regarding the comparison between the reactivity of the different sera against Mpro versus RBD, it was found that the IgG reactivity against the protease Mpro was comparable, or sometimes even stronger, to the reactivity against RBD, however, no differences were noticed between the RBD recombinant proteins expressed in either mammalian cells or baculovirus (FIG. 19E). To further validate the assay, additional controls were performed such as monitoring the background in plates with no viral antigen coating and testing sera collected before the COVID-19 pandemic (FIG. 7).

The specificity of the recognition was obvious in coating titration experiments and sera titration (FIG. 4).

### Specificity and Sensitivity/ROC Analysis of Above Data

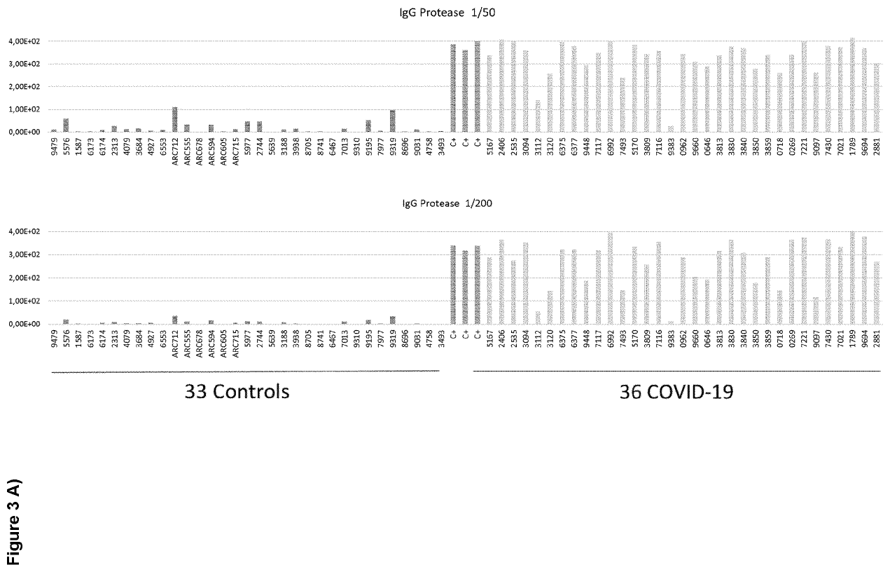

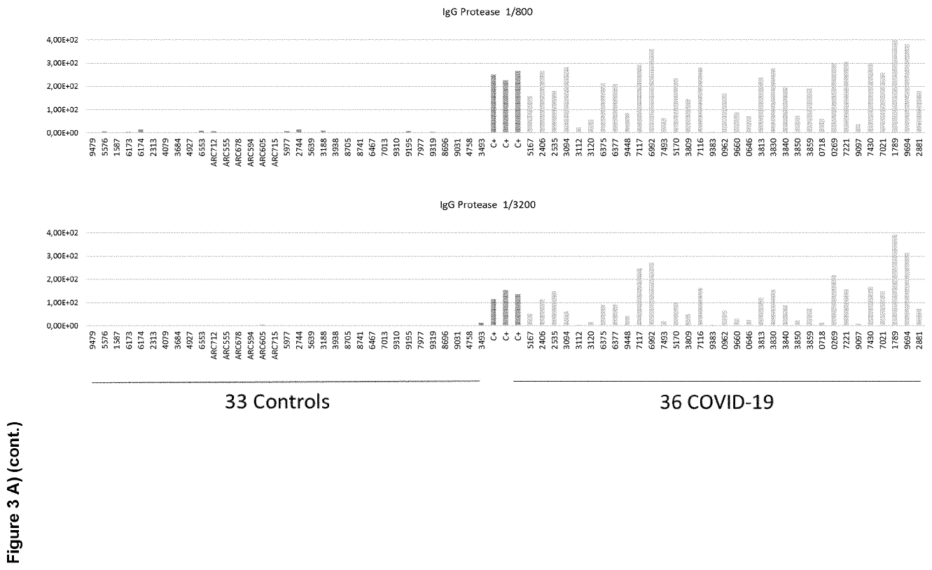

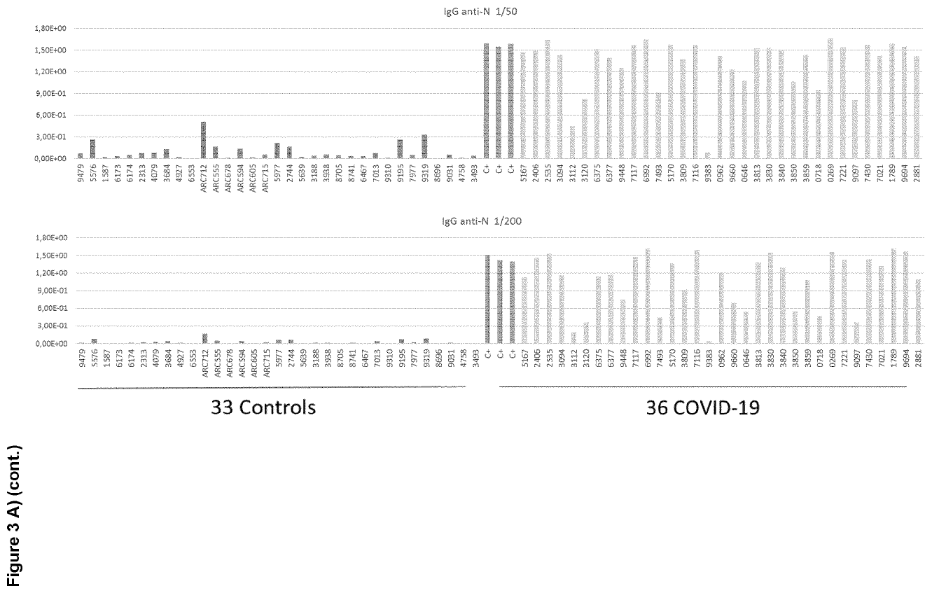

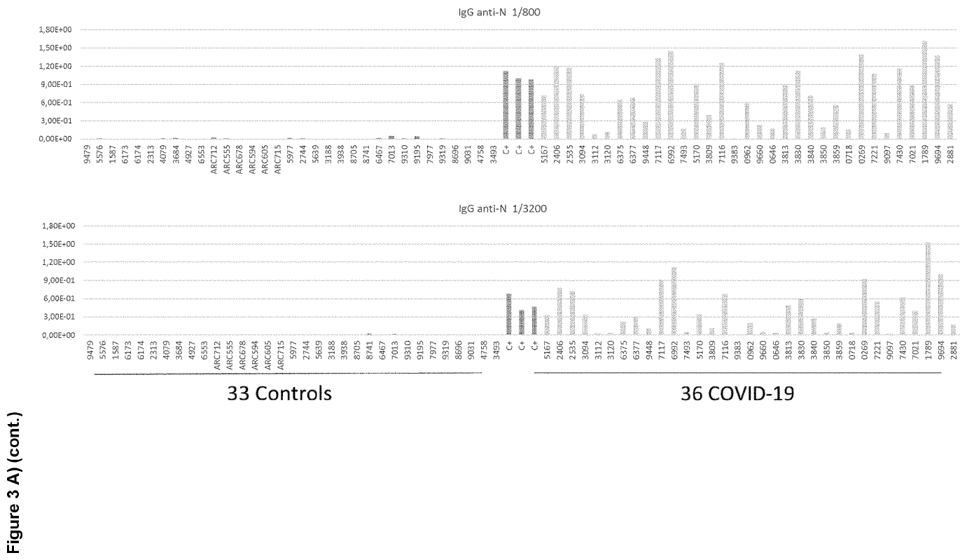

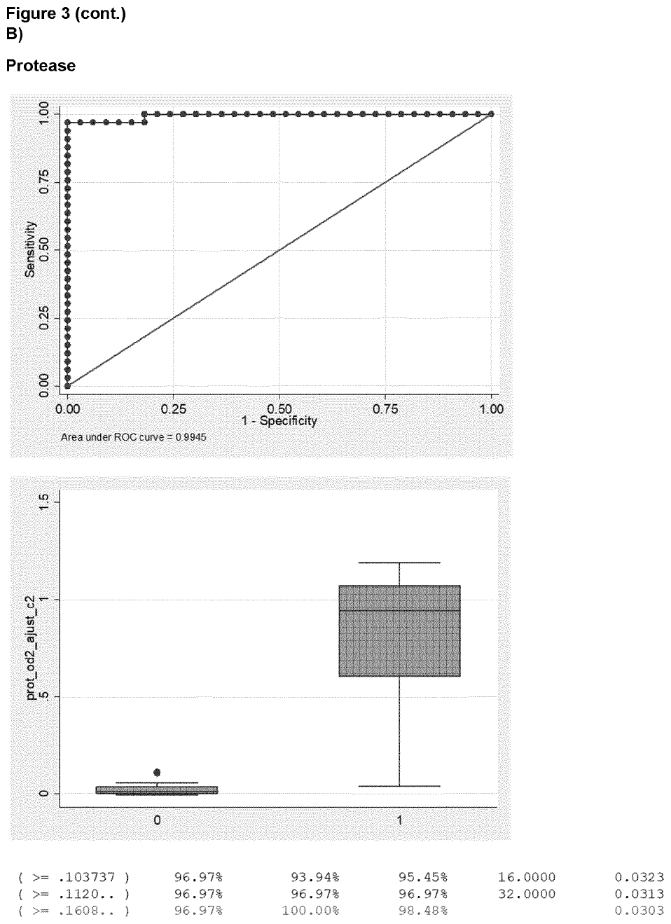

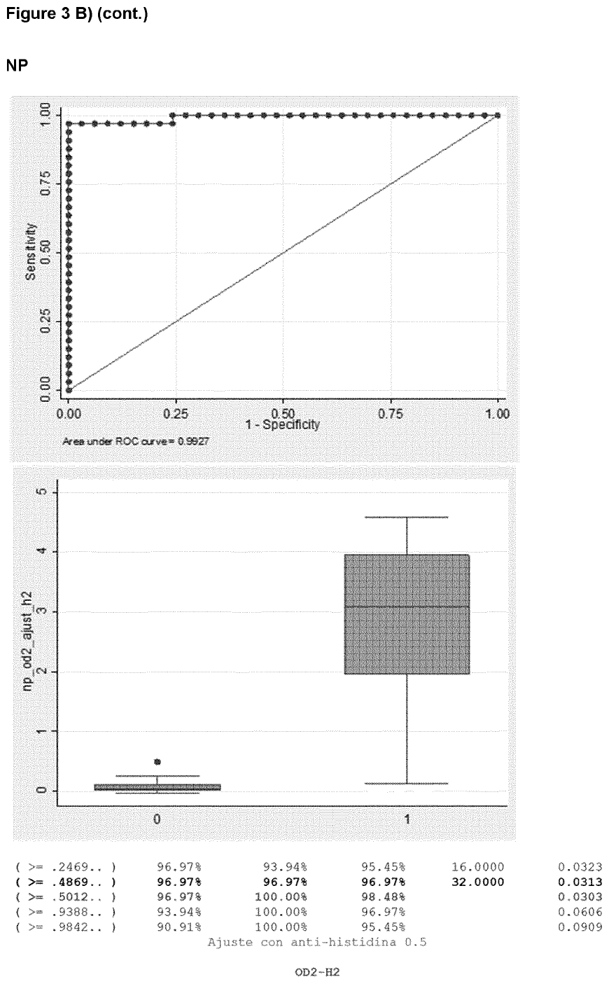

ELISAs based on both antigens (NP and protease-His) detect IgG antibodies with high sensitivity and specificity, the assay based on the protease as antigen at least as sensitive and specific as NP (FIG. 3).

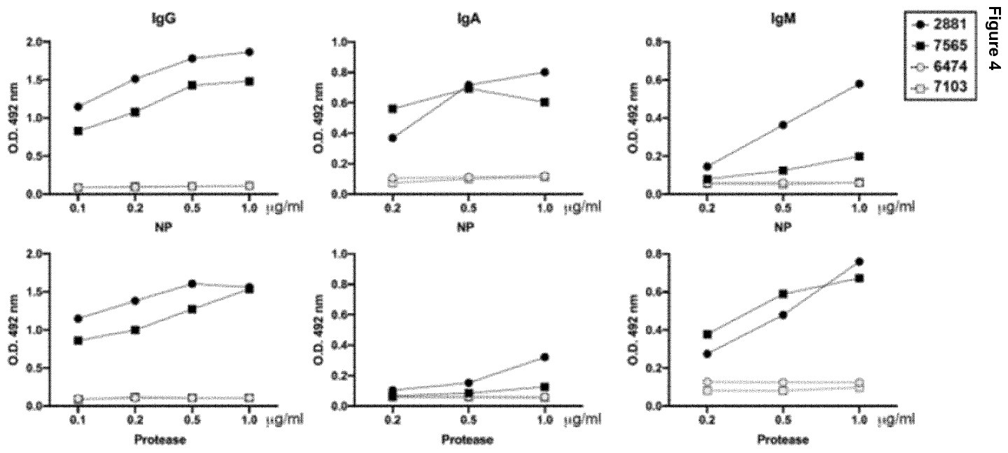

The comparison, as shown in FIG. 5, using serum samples, of ELISAs based on the nucleoprotein, protease and mRBD antigens, suggests that assays using the N and P antigens may be more sensitive than those using mRBD.

Mpro-Specific Antibodies can be Detected in Serum of COVID-19 Patients by ELISA

Since this study evaluated, for the first time, whether coronavirus-infected individuals could generate an antibody response against the Cys-like protease, Mpro, other SARS-CoV-2 proteins, commonly used in serology tests, were produced, for comparison. Mpro and NP were expressed in E coli, and two different constructs of the Receptor Binding Domain (RBD) of the spike protein were used: one was expressed by transfection in mammalian cells (mRBD) and a second, produced by baculovirus infection of insect cells (iRBD-His). All the proteins, except mRBD, had a histidine-tag and they were purified on Ni²⁺-NTA columns followed by size exclusion chromatography (FIG. 19A-D).

Detection of SARS-CoV-2 Mpro-Specific Antibodies Identifies COVID-19 Seropositive Individuals with High Specificity and Sensitivity

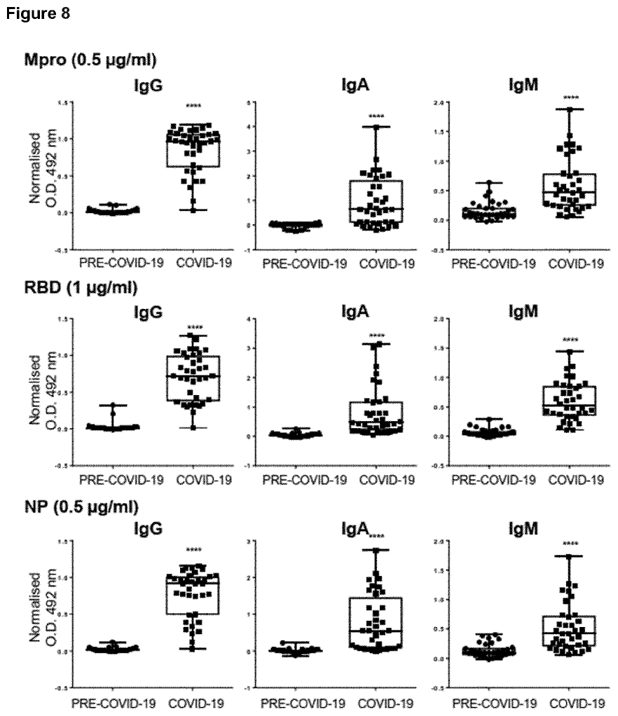

A cohort of 36 COVID-19 patients (PCR+) and 33 healthy donors was recruited at La Princesa University Hospital, Madrid (Table 1) and ELISA assays were performed to detect Mpro-, as well as RBD- and NP-, specific antibodies of the IgG, IgA and IgM subclasses in sera (FIG. 8).

TABLE 1
Patient demographic and clinical data.

| | | | N = 36 | % |
|---|---|---|---|---|
| Gender | Male | | 21 | 58 |
| | Female | | 15 | 42 |
| Age | <35 | | 7 | 19 |
| | 35-60 | | 18 | 50 |
| | >60 | | 11 | 31 |
| Time from symptoms onset | <15 days | | 2 | 6 |
| to sample collection | 15-30 days | | 13 | 36 |
| | 31-45 days | | 14 | 39 |
| | >45 | | 7 | 19 |
| Hospitalization | Yes | Ward | 4 | 11 |
| | | ICU | 6 | 17 |
| | No | | 26 | 72 |
| Fever | | | 31 | 86 |
| Shivers | | | 23 | 64 |
| Headache | | | 22 | 61 |
| Confusion | | | 6 | 17 |
| Conjunctival congestion | | | 5 | 14 |
| Nasal congestion | | | 18 | 50 |
| Rhinorrhea | | | 16 | 44 |
| Anosmia | | | 16 | 44 |
| Ageusia | | | 18 | 50 |
| Odynophagia | | | 14 | 39 |
| Dry cough | | | 19 | 53 |
| Productive cough | | | 9 | 25 |
| Dyspnea | | | 21 | 58 |
| Chest pain | | | 12 | 33 |
| Tonsillitis | | | 3 | 8 |
| Adenopathies | | | 4 | 11 |
| Nausea/vomiting | | | 10 | 28 |
| Diarrhea | | | 16 | 44 |
| Skin rash | | | 2 | 6 |
| Acrocyanosis | | | 1 | 3 |
| Myalgia/arthralgia | | | 24 | 67 |
| Asthenia | | | 27 | 75 |
| Weight loss | | | 20 | 56 |
| Thrombotic events | | | 2 | 6 |
| Comorbidities (HTN, | | | 17 | 47 |
| DM, COPD, obesity, cancer) | | | | |

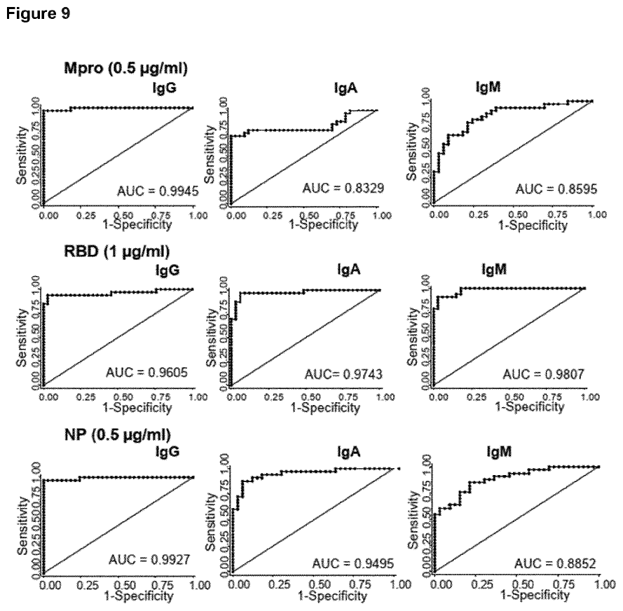

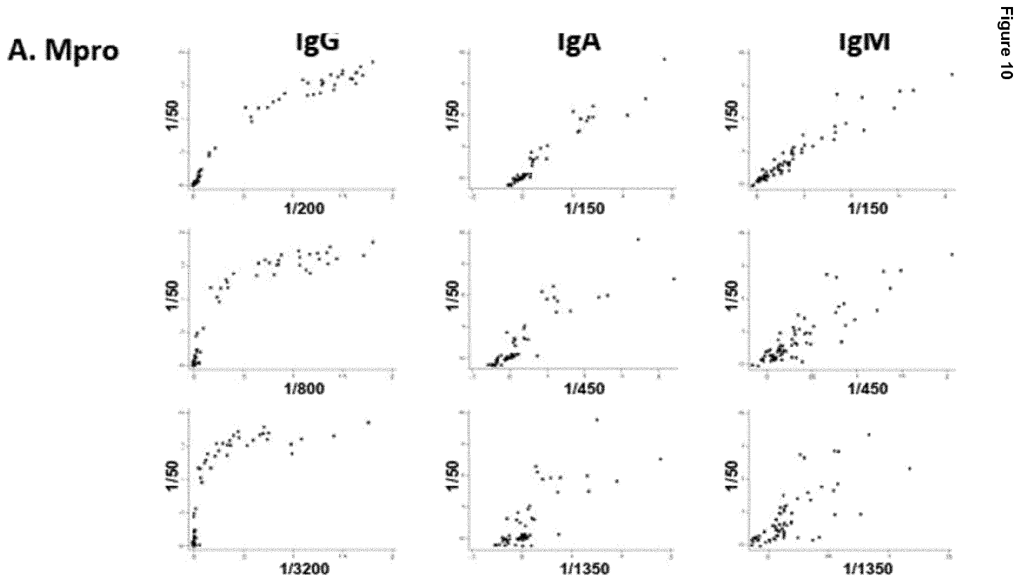

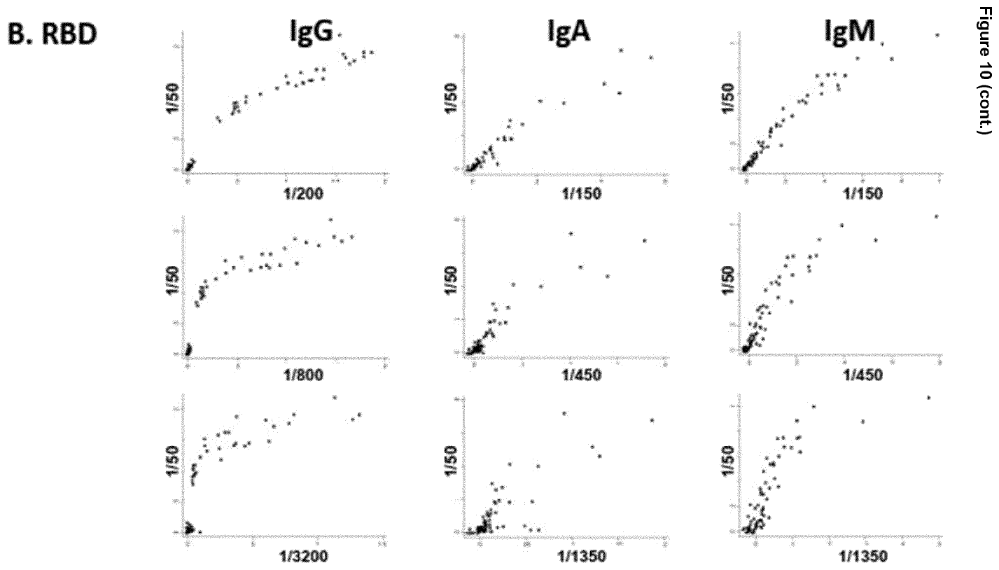

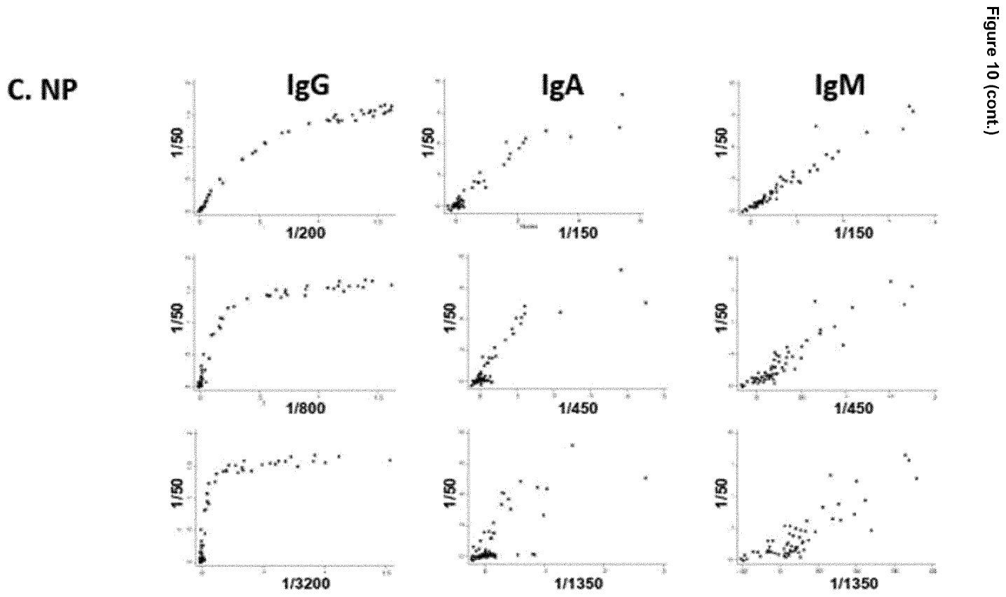

Titration of the serum samples was carried out over a dilution range of 1/50 to 1/3200, and these experiments showed that assay for seropositivity to all three antigens discriminated between COVID-19 positive and negative donors, as shown in dot plots comparing different dilutions (FIG. 10). FIG. 8 summarises the absorbance data from all the sera samples. To estimate the cut-off value, the sensitivity, and the specificity parameters for each antigen/Ig isotype pair, receiver operating characteristic (ROC) analyses were performed (Table 2, FIG. 9). The best area under the curve (AUC) values were obtained with the measurement of IgG antibodies specific for Mpro and NP (AUCs=0.9945 and 0.9927, respectively). The sensitivity and specificity were above 90% for detection of IgG antibodies of the three proteins tested, with values of sensitivity and specificity for Mpro of 97% and 100% respectively. AUC values above 0.85 were obtained for the other isotypes (IgA, IgM). Measurement of anti-IgA antibodies appeared to discriminate less accurately between pre-COVID-19 sera and COVID-19 sera, however, this is not due to a lack in sensitivity for this isotype. Instead, because background levels with IgA were very low and the signal clearly positive in some patients, the lack of detection suggests that certain COVID-19-positive patients have circulating IgA while other COVID-19-positive patients lack IgA in peripheral blood. Whether the presence of IgA in periphery has any relationship with clinical aspects needs to be explored further in larger cohorts of patients.

TABLE 2
AUC, cut-off, sensitivity and specificity

| Antigen | Isotype | AUC | Cut-off | Sensitivity | Specificity |
|---|---|---|---|---|---|
| RBD | IgG | 0.961 | 0.232 | 94% | 97% |
| | IgA | 0.974 | 0.112 | 97% | 94% |
| | IgM | 0.981 | 0.203 | 91% | 97% |
| Mpro | IgG | 0.994 | 0.161 | 97% | 100% |
| | IgA | 0.833 | 0.130 | 73% | 100% |
| | IgM | 0.859 | 0.237 | 79% | 79% |
| NP | IgG | 0.993 | 0.127 | 97% | 100% |
| | IgA | 0.949 | 0.066 | 88% | 94% |
| | IgM | 0.885 | 0.341 | 76% | 85% |

AUC, area under the curve;
RBD, Receptor Binding Domain;
Mpro, cysteine-like protease;
NP, nucleoprotein

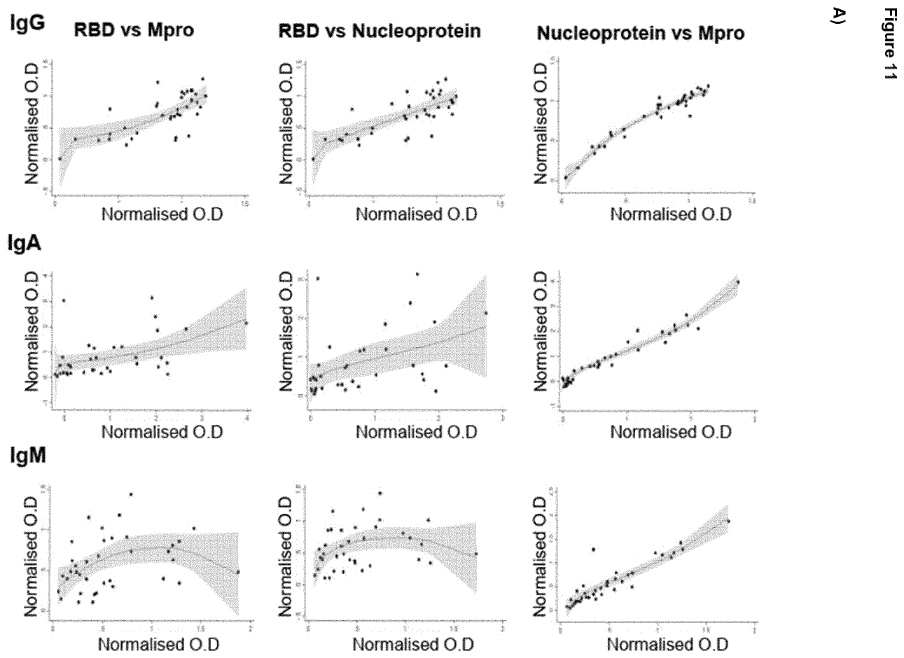

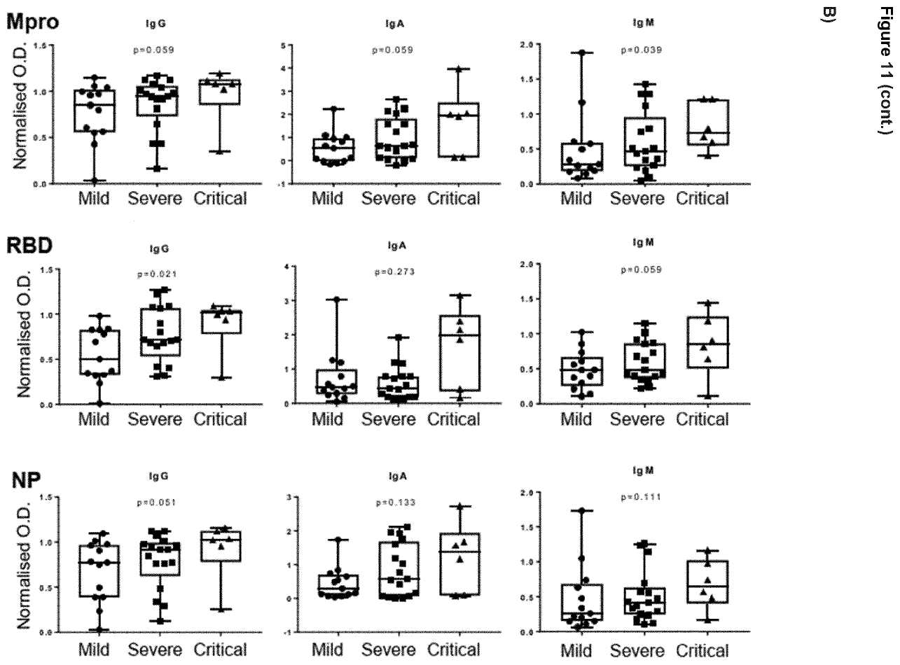

Comparison between proteins showed some heterogeneity in the capacity of different donors to produce antibodies, especially for IgM and IgA subclasses. Non-linear polynomial regression showed a better correlation between the detection of antibodies against NP and Mpro compared to NP and RBD or Mpro and RBD (FIG. 11A). Only one COVID-19 donor failed to make a full antibody response.

Further analyses were performed to explore the correlations between the titres of the different antibodies in serum and clinical parameters. Interestingly, a trend for higher titre antibody responses was found in patients with more severe disease (FIG. 11B), being more pronounced for IgM against Mpro and IgG against RBD. However, several other variables also contributed to the heterogeneity in antibody response, mainly age and time since the onset of symptoms (Table 3). After adjustment for these possibly confounding factors, IgA anti-RBD was observed to be significantly higher in critical patients compared to patients with mild disease. In addition, critical patients showed a trend to higher IgM and IgA anti-Mpro titres than patients with mild COVID-19. Furthermore, intense IgM and IgA responses against the three proteins were significantly associated with higher serum IL-6 levels (data not shown).

TABLE 3
Variables that explain heterogeneity in Ab response
against three proteins of SARS-CoV-2 virus.

| | IgG | | IgA | | IgM | |
|---|---|---|---|---|---|---|
| | β | | β | | β | |
| | Coeff. | p | Coeff. | p | Coeff. | p |
| Mpro | | | | | | |
| Age (y) | 0.010 | 0.013 | 0.021 | 0.085 | NRM | — |
| Time since | NRM | — | −0.033 | 0.010 | −0.014 | 0.005 |
| symptoms onset (d) | | | | | | |
| Severity | | | | | | |
| Mild | Ref. | — | Ref. | — | Ref. | — |
| Severe | −0.004 | 0.970 | 0.422 | 0.196 | 0.189 | 0.220 |
| Critical | −0.013 | 0.935 | 0.804 | 0.092 | 0.364 | 0.073 |
| RBD | | | | | | |
| Age (y) | 0.007 | 0.083 | NRM | — | 0.008 | 0.037 |
| Time since | NRM | — | −0.016 | 0.058 | −0.009 | 0.006 |
| symptoms onset (d) | | | | | | |
| Severity | | | | | | |
| Mild | Ref. | — | Ref. | — | Ref. | — |
| Severe | 0.149 | 0.195 | −0.032 | 0.904 | 0.087 | 0.434 |
| Critical | 0.198 | 0.243 | 1.014 | 0.004 | 0.207 | 0.203 |
| NP | | | | | | |
| Age (y) | 0.009 | 0.039 | 0.016 | 0.092 | NRM | — |
| Time since | NRM | — | −0.024 | 0.002 | −0.011 | 0.016 |
| symptoms onset (d) | | | | | | |
| Severity | | | | | | |
| Mild | Ref. | — | Ref. | — | — | — |
| Severe | 0.023 | 0.849 | 0.379 | 0.144 | 0.109 | 0.451 |
| Critical | 0.048 | 0.789 | 0.494 | 0.191 | 0.246 | 0.195 |

Coeff., coefficient; NRM, not relevant for the model.

Therefore, the use of SARS-CoV-2 Mpro, in combination with other antigens already described for serology tests, provided outstanding specificity and sensitivity for patient identification. IgG titrated further than IgA or IgM indicating that, as expected, the IgG subclass is more abundant in serum. Assay for IgM antibodies had a lower signal/noise ratio and, in many of the SARS-CoV-2 negative sera a significant background could be observed for IgM. In contrast, SARS-CoV-2-specific IgA antibodies were not detected in healthy donors, but were clearly present in 27 out of the 36 sera tested from COVID-19 patients.

### Kinetics of SARS-CoV-2 Antibodies Response

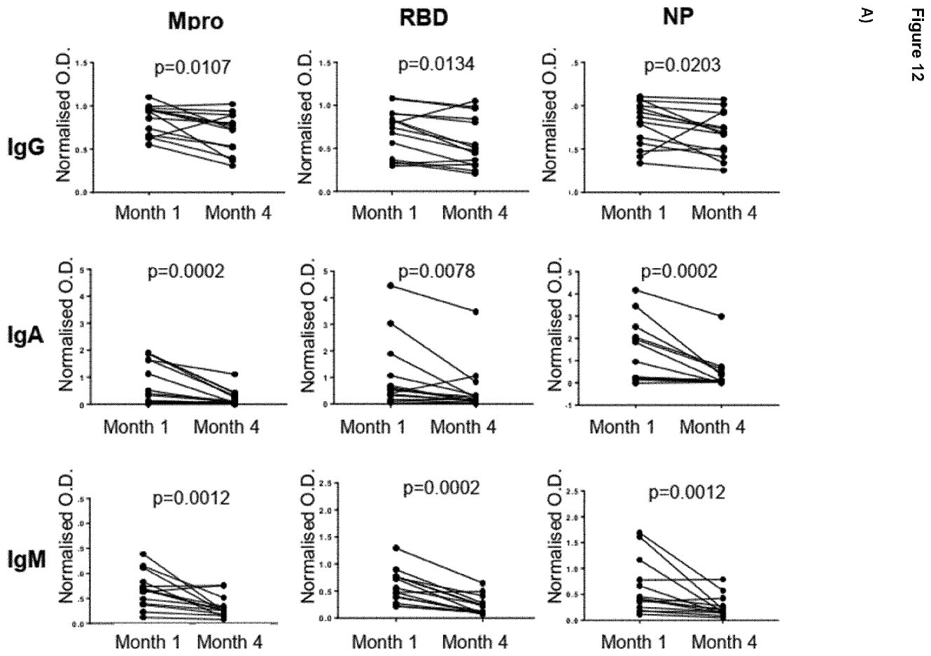

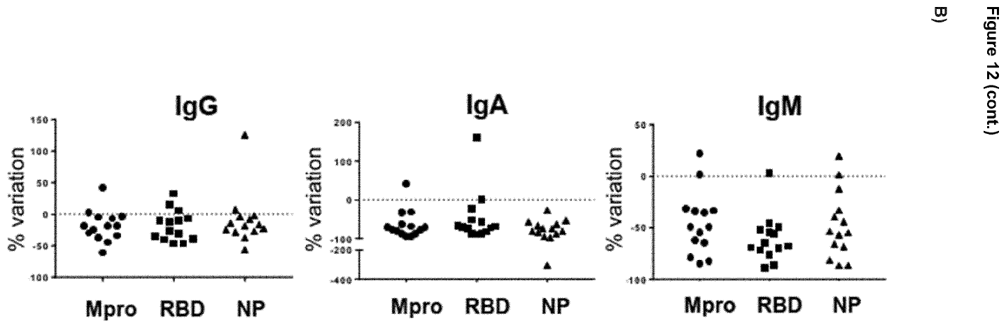

In order to compare the kinetics of Mpro antibody response with that of other SARS-CoV-2 proteins a follow up of 14 patients was performed, selecting 7 patients with a high titre the first month after the onset of symptoms and 7 patients with a medium-low titre at the same time point. Antibody levels were compared for each donor (FIG. 12A) and the percentage of change was also calculated (FIG. 12B). IgG concentration decreased slightly in most of the patients, however, antibodies against the three proteins could still be detected 4 months after the onset of symptoms. In contrast, IgA and IgM levels clearly decreased and, in certain patients, reached background levels.

One patient showed a marked increase in IgG in the sample obtained four months after the onset of symptoms. In this case, the first sample was obtained on the first week of symptoms, probably when the immune response was still not fully activated. The patient had then a severe disease, explaining the increase in antibody levels later in time.

Mpro-Specific IgG Antibodies are Detected in Saliva from COVID-19 Patients

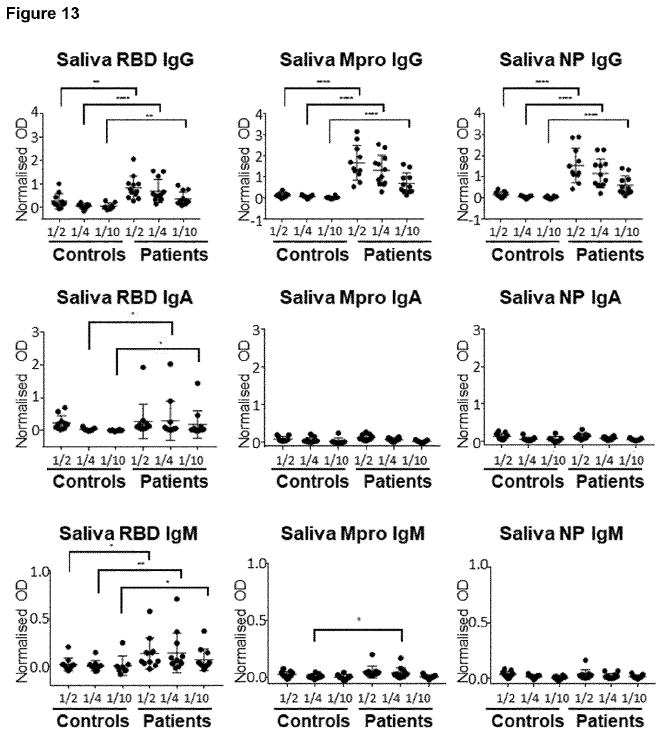

Saliva samples were collected from 11 healthy donors and 12 COVID-19 patients at the University Hospital La Princesa (Madrid) and tested in ELISA assays over a range of dilutions (1/2 to 1/10). IgG recognizing the three viral antigens tested could be observed in COVID-19 patients, with the strongest responses being those specific for the viral protease Mpro (FIG. 13). IgA and IgM responses were detected in only one of the COVID-19 infected individuals. This saliva sample was collected 59 days after the confirmation of a SARS-CoV-2 positive PCR test and the patient had very mild disease.

### Example 3

### Materials and Methods

### Patient Selection, Samples and Institutional Review Board Permits

Experiments were carried out following the ethical principles established in the 1964 Declaration of Helsinki and its later amendments or comparable ethical standards. Patients (or their representatives) were informed about the study and gave a written informed consent. This study used samples from several hospitals. For optimization experiments, samples from the research project “Immune response dynamics as predictor of COVID-19 disease evolution. Implications for therapeutic decision-making” approved by La Princesa Health Research Institute Research Ethics Committee (register #4070) were used; samples and data from patients with severe vs mild disease were provided by the Biobank Hospital Universitario Puerta de Hierro Majadahonda (HUPHM)/Instituto de Investigación Sanitaria Puerta de Hierro-Segovia de Arana (IDIPHISA) (PT17/0015/0020 in the Spanish National Biobanks Network), they were processed following standard operating procedures with the appropriate approval of the Ethics and Scientific Committees. For comparison of disease severity, 29 COVID-19 patients, diagnosed by PCR, were recruited. 14 patients, classified as mild disease or asymptomatic, did not require treatment after diagnosis. 15 patients, classified as severe disease, required ICU hospitalization (Table 4). Plasma samples were obtained 33-40 days after diagnostic PCR and, separated by blood centrifugation after collection in EDTA tubes, 15 plasma samples collected from healthy blood donors before June 2019 (PRE-COVID-19) in the Puerta de Hierro hospital biobank, were used as negative controls.

TABLE 4
Patient demographic and clinical data for severity study

| | | | N = 29 | % |
|---|---|---|---|---|
| Gender | Male | | 25 | 86 |
| | Female | | 4 | 14 |
| Age | 40-60 | | 19 | 66 |
| | 61-70 | | 5 | 17 |
| | >71 | | 5 | 17 |
| Time from symptoms onset | <15 days | | 3 | 10 |
| to sample collection | 15-30 days | | 8 | 28 |
| | 31-45 days | | 4 | 14 |
| | >45 | | 9 | 48 |
| Hospitalization | Yes | ICU | 15 | 55 |
| | No | | 14 | 45 |
| Fever | | | 16 | 55 |
| Rhinorrhea | | | 1 | 3 |
| Anosmia | | | 4 | 14 |
| Ageusia | | | 3 | 10 |
| Odynophagia | | | 2 | 7 |
| Dry cough | | | 11 | 38 |
| Productive cough | | | 3 | 10 |
| Dyspnea | | | 11 | 38 |
| Pneumonia | | | 13 | 45 |
| Fatigue, myalgia, anorexia | | | 14 | 48 |
| Pulmonary infiltrate | | | 13 | 45 |
| Gastrointestinal symptoms | | | 0 | 0 |
| Cutaneous lesions | | | 0 | 0 |
| Multiorgan dysfunction | | | 5 | 17 |
| Thrombotic events | | | 2 | 7 |
| Comorbidities (HTN, DM, | | | 7 | 24 |
| COPD, obesity, cancer) | | | | |
| Required mechanical | | | 13 | 45 |
| ventilation | | | | |
| Death | | | 2 | 7 |

ICU (intensive care unit), HTN (hypertension), DM (diabetes mellitus), COPD (chronic obstructive pulmonary disease).

15 vaccinated (Pfizer BioNTech) individuals were recruited at the Centro de Hemoterapia y Hemodonación de Castilla y León (ChemCyL) for comparison with SARS-CoV-2 infected patients (Table 5).

TABLE 5
Patient demographic for vaccination study

| | | N = 15 | % |
|---|---|---|---|
| Gender | Male | 5 | 33.3 |
| | Female | 10 | 66.6 |
| Age (25-56) | 25-35 | 4 | 26.6 |
| | 36-45 | 4 | 26.6 |
| | >46 | 7 | 46.6 |
| Days from first | 2 days | 1 | 6.6 |
| dose (2-35) | 10-20 days | 5 | 33.3 |
| | 21-30 days | 6 | 40.0 |
| | >31 | 3 | 20.0 |
| Patients having | Y | 9 | 60.0 |
| received 2^{nd} dose | N | 6 | 40.0 |

These samples were obtained as part of the project “Development of serological assays for detection of viral antigens (SARS-COV2)”. The protocol was approved by the Bioethics Committees: CElm Área de Salud Valladolid Este, Hospital Clínico Universitario de Valladolid, with the number/BIO 2020-98-COVID.

CPD respiratory panel human plasma samples were obtained from a commercial source (BioIVT—West Sussex, United Kingdom).

### Expression of the SARS-CoV-2 Cys-Like Protease (Mpro), Nucleocapsid (NP), Spike (S) and RBD Proteins

Recombinant SARS-CoV-2 proteins were expressed with a histidine tag. Cys-like protease (Mpro) and nucleocapsid (NP) proteins constructs were expressed in the E. coli strain BL21 Star (DE3) pLysS (ThermoFisher) and purified as described above.

Recombinant cDNAs coding for soluble S (residues 1 to 1208) and RBD (332 to 534) proteins were cloned in the pcDNA3.1 vector for expression in HEK-293F cells using standard transfection methods. The two constructs contained the S signal sequence at the N-terminus, and a T4 fibritin trimerization sequence, a Flag epitope and an 8×His-tag at the C-terminus. In the S protein, the furin-recognition motif (RRAR) was replaced by the GSAS sequence and it contained the A942P, K986P and V987P substitutions in the S2 portion. Proteins were purified by Ni-NTA affinity chromatography from transfected cell supernatants and they were transferred to 25 mM Hepes-buffer and 150 mM NaCl, pH 7.5, during concentration.

Bead based flow cytometry assay for detection of antibodies to SARS-CoV-2

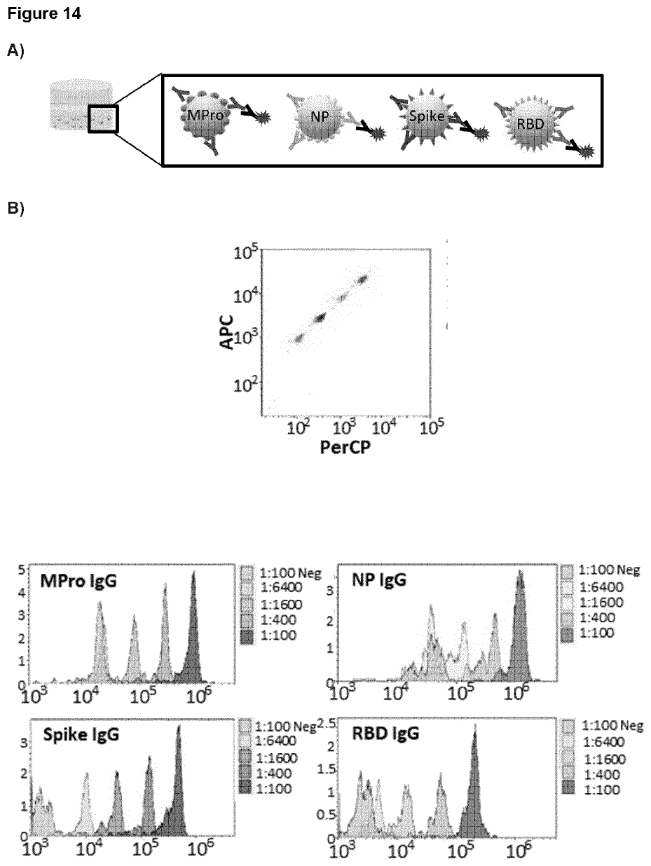

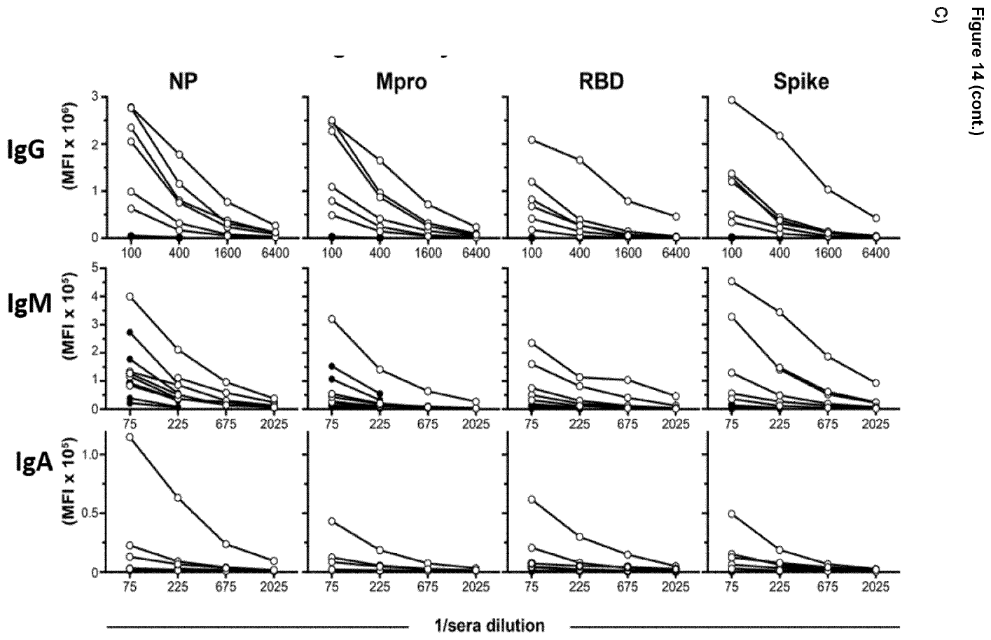

10⁶ magnetic fluorescent beads, with a mean diameter 5.5 μm and high density carboxyl functional groups on the surface (QuantumPlex™ M COOH—Bangs Laboratories, Inc.), were covalently coupled with 30 μg of viral protein through their primary amines by two-step EDC/NHS protocol. Beads were resuspended in a solution of PBS containing 1% casein and a stabilizer (Biorad 1×PBS blocker). To distinguish the beads coated with different antigens, different fluorescence intensity combinations in the APC and PerCP channels were used (FIG. 14).

Beads were incubated with either rabbit anti-His-tag antibody (Proteintech Group) or plasma from patients or healthy donors in a final volume of 50 μl in 96-well-plates (Nunc™ MicroWell™ 96-Well, Thermo Fisher Scientific) using the dilutions indicated in each experiment. Patient plasma samples were diluted in PBS-casein (Biorad, 1×PBS blocker), and incubated with the beads for 40 min at room temperature under agitation. Beads were washed three times by addition of PBS, placing the tubes or plates on a magnet (MagneSphere® Mag. Sep. Stand 12-hole, 12×75 mm, Promega; Handheld Magnetic Separator Block for 96 well plate, Merck, Millipore) and decantation of supernatant.

To visualize antibody bound to antigen-coated beads, either PE-conjugated anti-rabbit antibody (0.25 μg/ml, Southern Biotech), PE-conjugated anti-human IgG and IgM, or FITC-conjugated anti-human IgA antibody (Immunostep S.L.) were added (30 μL/well) and incubated for 20 minutes at room temperature under agitation. After three washes, data were acquired by flow cytometry using either CytoFLEX or Cytomics FC 500 (Beckman Coulter).

For large screenings performed in different days, data were normalized to the values of a positive control serum included in every assay.

### ELISA for Detection of Antibodies to SARS-CoV-2

ELISA assays for detection of antibodies directed against the four SARS-CoV-2 antigens were carried out as described above.

### Statistical Analysis

To assess the prediction capacity of the new methodology, an algorithm was built using Scikit-learn python package (F P, G V, A G, V M, B T, O G, et al. Scikit-learn: Machine Learning in Python. Journal of Machine Learning Research. 2011; 12:2825-30). Samples were stratified and randomly spliced into a training and a test set. The training samples were used to fit a random forest classifier which then predicted the healthy vs disease category of unseen test samples (1/7 of total samples). This was repeated n=10,000 times. For each patient, accuracy was calculated as the proportion of correct predictions divided by the number of predictions made. As a complementary approach, a mean Receiver Operating Characteristic (ROC) curve was built for the random forest classifier by stratified 15-fold cross-validation, using the smaller set (2-3 samples) to train the model and then predicting the remaining ones.

For heatmap representation, each variable was scaled to a range (0,1) using the MixMaxScaler command from Scikit-learn and visualized using heatmap command from seaborn python packages. For Principal Component Analysis, each variable was scaled as described, and the PCA command from Scikit-learn was used to fit and transform the data. Principal components up to a 95% of accumulated explained variance were saved.

Comparison between severe and mild patients in each variable was performed by multiple t-tests followed by False Discovery Rate (1%) correction by two-stage step-up method in Graph Pad Prism 8 Software (GraphPad Software, USA, www.qraphpad.com).

### Results

### Basic Bead-Assisted Multi-Antigen Serological Assay Using Flow Cytometry

The NP, S and RBD proteins of Coronaviruses have been widely used in single-antigen serological assays for SARS and MERS-caused diseases. However, the use of these antigens in combination with the immunogenic Mpro, can more fully describe the magnitude and duration of the immune response in SARS-CoV-2-infected patients. In order to facilitate comprehensive characterization of COVID-19 patients with a high throughput approach, a multi-antigen assay was developed with several viral antigens immobilised on fluorescent beads, to allow flow cytometry detection of the multiple antibodies generated during SARS-CoV-2 infections.

As depicted in FIG. 14A, the assay uses fluorescent magnetic beads coated with SARS-CoV-2 antigens. Each protein was immobilised on a bead population with a particular fluorescence intensity in the red channel (e.g. APC/PerCP). This permits simultaneous detection of antibodies to different antigens in a single test tube or well. After incubation with patient plasma, one or several secondary antibodies conjugated to different fluorophores, such as FITC or PE, were used for identification of the IgG, IgA and IgM immunoglobulins bound to the viral antigens. In most experiments, combinations of anti IgM-PE and IgA-FITC were used. IgG and IgA were also combined with good results. Thus, the data for each antigen-specific Ig could be determined in a single reaction, by using three different fluorophores. Specifically, in several experiments the FITC, PE and PE-Cyanine7 fluorochrome combination was tested for the detection of IgG, IgA and IgM respectively.

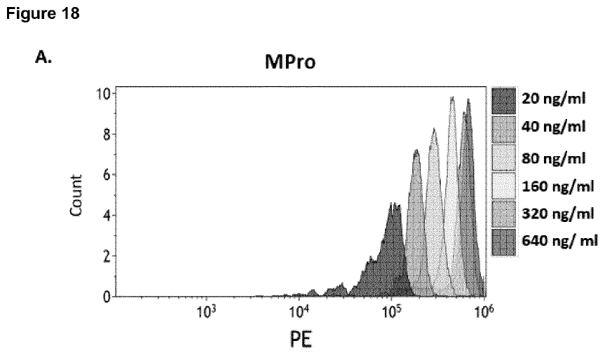

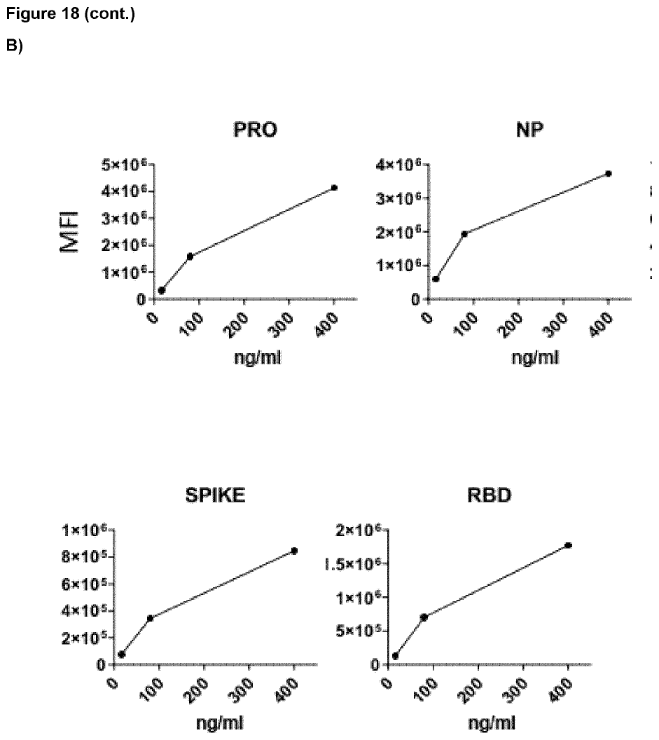

Initially, to define the detection limits and the amount of antibody binding, titration experiments varying the amount of beads (not shown) and the concentration of anti-His-tag antibody were performed (FIG. 18). These trial experiments allowed estimation of the signal for a known concentration of antibody and the data suggested that the new methodology could provide good sensitivity. Indeed, since all the antigen constructs have only a single His-tag, it would be expected that the use of plasma containing a polyclonal mixture of antibodies binding multiple epitopes would provide more signal and further increase sensitivity.

The anti-His signal obtained for the S protein was lower compared to other viral antigens but did not affect detection in patient plasma. The lower detection of S by His-tag antibody was likely due to a lower molar amount of S than NP, Mpro or RBD bound to the beads. This was expected because S molecular weight (˜180 KDa) is at least four times higher than the other antigens (25-40 KDa). Sera analysis allowed a very good separation of control and convalescent samples in a wide range of dilutions (FIG. 14B,C). Thus, the use of magnetic beads and flow cytometry is a suitable technique for the serological analyses.

Multi-Antigen Bead-Assisted Flow Cytometry Identifies COVID-19 Patients with 100% Confidence

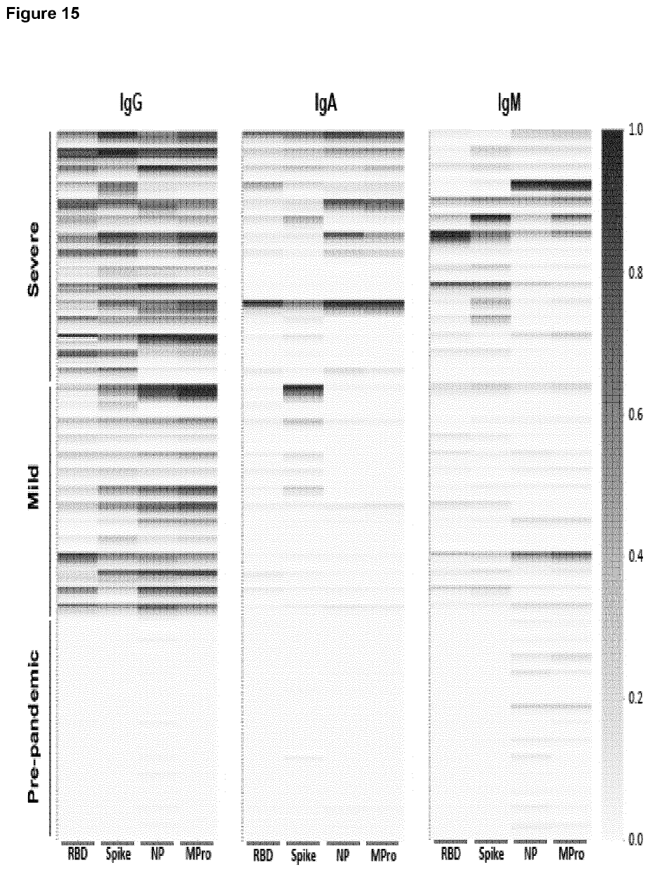

The sensitivity and specificity of the new method was evaluated by testing for the presence of antibodies against four SARS-CoV-2 antigens (S, RBD, NP, Mpro) in 44 plasma samples, including 29 COVID-19 patients, 14 of them with mild disease and 15 with severe disease. Each plasma sample was tested over a range of dilutions (1:100 to 1:5400) for three Ig isotypes (IgA, IgG, IgM). Initial analysis using heat map representations of the data (FIG. 15), shows a clear difference between the signal obtained for IgG antibodies against the four antigens between healthy controls and COVID-19 patients. As expected, although, IgA and IgM SARS-CoV-2-specific antibodies were detected in multiple patients, they were not present in all the sera tested. While IgM had a higher background, IgA provided very clean and specific data.

Machine learning techniques were used to assess the specificity and sensitivity of this novel methodology. A random forest classifying algorithm was developed to evaluate the prediction capacity of seronegative versus COVID seropositive individuals when data were generated by ELISA and by FACS, comparing both single-antigen and multi-antigen techniques. When only the data generated for IgG by FACS were analysed, all the patients were correctly classified in 100% of the multi-antigen repetitions, except for two that were correctly classified 99.93% and 98.24% of the times (Table 6). Combining data for the four antigens and 4 dilutions for IgG provides an overall prediction capacity of 99.94% true positive rate and 100% true negative rate. True positive rates near to 100% were also obtained when only three antigens (RBD, S and Mpro) and one dilution were analysed, highlighting the predictive power of the technique [IgG 1/100 99.87; IgG 1/200 99.98; IgG 1/600 98.94; IgG 1/1800 x=99.98]. In all cases, true negative rate was always 100%. Individual antigens by ELISA had slightly lower prediction values. Thus, the use of a single test including three antigens, one isotype detection (IgG) and one dilution results in accurate classification of patients, facilitating large screenings.

TABLE 6
Prediction accuracy of healthy vs COVID samples*

| | | | | | | FACS | FACS | | | |
|---|---|---|---|---|---|---|---|---|---|---|
| Samples | | ELIS | | | | Multiantigenic | RBD-Spike-Pro | | | |
| Donor | Status | RBD | Spike | NP | Pro | 1:100-1:800 | 1:100 | 1:200 | 1:600 | 1:1800 |
| 8 | Covid-19 | 100 | 100 | 100 | 100 | 100 | 100 | 100 | 100 | 100 |
| 11 | Covid-19 | 100 | 100 | 100 | 100 | 100 | 100 | 100 | 100 | 100 |
| 14 | Covid-19 | 100 | 100 | 100 | 100 | 100 | 100 | 100 | 100 | 100 |
| 15 | Covid-19 | 100 | 100 | 100 | 100 | 100 | 100 | 100 | 100 | 100 |
| 16 | Covid-19 | 100 | 100 | 100 | 100 | 100 | 100 | 100 | 100 | 100 |
| 18 | Covid-19 | 100 | 100 | 100 | 100 | 100 | 100 | 100 | 100 | 100 |
| 21 | Covid-19 | 100 | 100 | 100 | 100 | 100 | 100 | 100 | 100 | 100 |
| 97 | Covid-19 | 100 | 100 | 100 | 100 | 100 | 100 | 100 | 100 | 100 |
| 102 | Covid-19 | 100 | 100 | 100 | 100 | 100 | 100 | 100 | 100 | 100 |
| 103 | Covid-19 | 100 | 100 | 100 | 100 | 100 | 100 | 100 | 100 | 100 |
| 106 | Covid-19 | 100 | 100 | 100 | 100 | 100 | 100 | 100 | 100 | 100 |
| 107 | Covid-19 | 100 | 100 | 100 | 100 | 100 | 100 | 100 | 100 | 100 |
| 109 | Covid-19 | 100 | 100 | 100 | 100 | 100 | 100 | 100 | 100 | 100 |
| 110 | Covid-19 | 100 | 100 | 100 | 100 | 100 | 100 | 100 | 100 | 100 |
| 128 | Covid-19 | 100 | 100 | 11.54 | 89.87 | 100 | 100 | 100 | 100 | 100 |
| 35 | Covid-19 | 100 | 100 | 100 | 100 | 100 | 100 | 100 | 100 | 100 |
| 57 | Covid-19 | 47.43 | 99.29 | 0 | 13.02 | 99.93 | 98.46 | 100 | 99.78 | 99.92 |
| 92 | Covid-19 | 100 | 100 | 100 | 100 | 100 | 100 | 100 | 100 | 100 |
| 64 | Covid-19 | 100 | 99.92 | 11.72 | 24.81 | 100 | 100 | 100 | 99.7 | 100 |
| 99 | Covid-19 | 100 | 100 | 100 | 100 | 100 | 100 | 100 | 100 | 100 |
| 100 | Covid-19 | 100 | 100 | 100 | 100 | 100 | 100 | 100 | 100 | 100 |
| 101 | Covid-19 | 100 | 100 | 100 | 100 | 100 | 100 | 100 | 100 | 100 |
| 129 | Covid-19 | 100 | 100 | 100 | 100 | 100 | 100 | 100 | 100 | 100 |
| 114 | Covid-19 | 99.37 | 98.68 | 100 | 100 | 98.24 | 97.53 | 99.57 | 68.26 | 99.49 |
| 89 | Covid-19 | 96.46 | 100 | 100 | 100 | 100 | 100 | 100 | 100 | 100 |
| 115 | Covid-19 | 100 | 100 | 100 | 100 | 100 | 100 | 100 | 100 | 100 |
| 121 | Covid-19 | 100 | 100 | 100 | 100 | 100 | 100 | 100 | 100 | 100 |
| 123 | Covid-19 | 100 | 100 | 100 | 100 | 100 | 100 | 100 | 100 | 100 |
| 130 | Covid-19 | 100 | 100 | 100 | 100 | 100 | 100 | 100 | 100 | 100 |
| 43 | Control | 100 | 100 | 100 | 100 | 100 | 100 | 100 | 100 | 100 |
| 36 | Control | 100 | 100 | 100 | 100 | 100 | 100 | 100 | 100 | 100 |
| 187 | Control | 100 | 100 | 100 | 100 | 100 | 100 | 100 | 100 | 100 |
| 94 | Control | 100 | 100 | 100 | 100 | 100 | 100 | 100 | 100 | 100 |
| 24 | Control | 100 | 100 | 100 | 100 | 100 | 100 | 100 | 100 | 100 |
| 32 | Control | 100 | 100 | 100 | 100 | 100 | 100 | 100 | 100 | 100 |
| 33 | Control | 100 | 100 | 100 | 100 | 100 | 100 | 100 | 100 | 100 |
| 75 | Control | 100 | 100 | 100 | 100 | 100 | 100 | 100 | 100 | 100 |
| 2 | Control | 100 | 100 | 100 | 100 | 100 | 100 | 100 | 100 | 100 |
| 8 | Control | 100 | 100 | 100 | 100 | 100 | 100 | 100 | 100 | 100 |
| 109 | Control | 100 | 100 | 100 | 100 | 100 | 100 | 100 | 100 | 100 |
| 125 | Control | 100 | 100 | 2.29 | 1.11 | 100 | 100 | 100 | 100 | 100 |
| 112 | Control | 100 | 100 | 100 | 100 | 100 | 100 | 100 | 100 | 100 |
| 80 | Control | 100 | 100 | 100 | 100 | 100 | 100 | 100 | 100 | 100 |
| 29 | Control | 100 | 100 | 100 | 100 | 100 | 100 | 100 | 100 | 100 |

*Samples were stratified and randomly spliced into training and test sets. The training samples were used to fit a random forest classifier which then predicted the healthy vs disease category of unseen test samples (1/7 of total samples). This was repeated n = 10,000 times. For each patient, accuracy was calculated as the proportion of correct predictions divided by the number of predictions made

### COVID-19 Patients Respond Differentially to the Four Viral Antigens

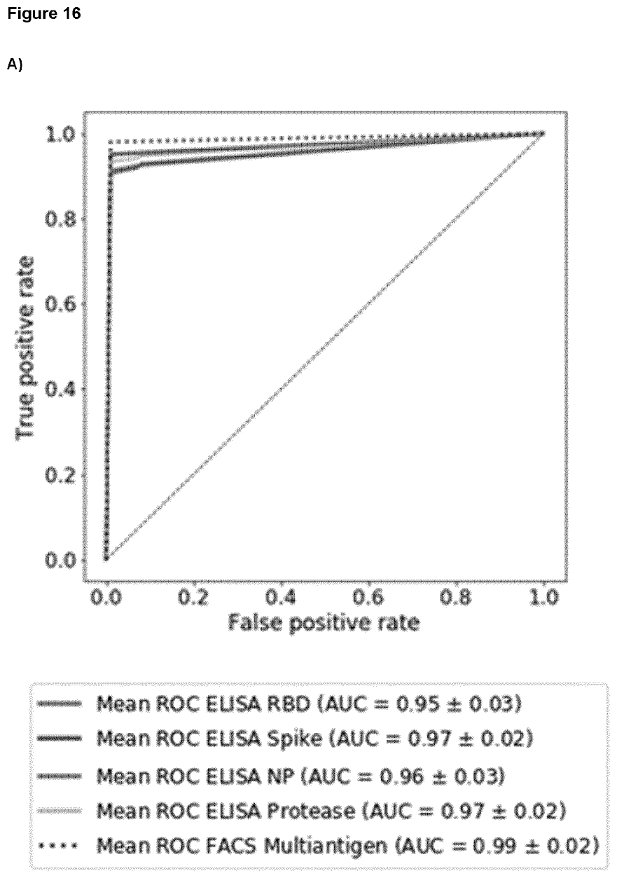

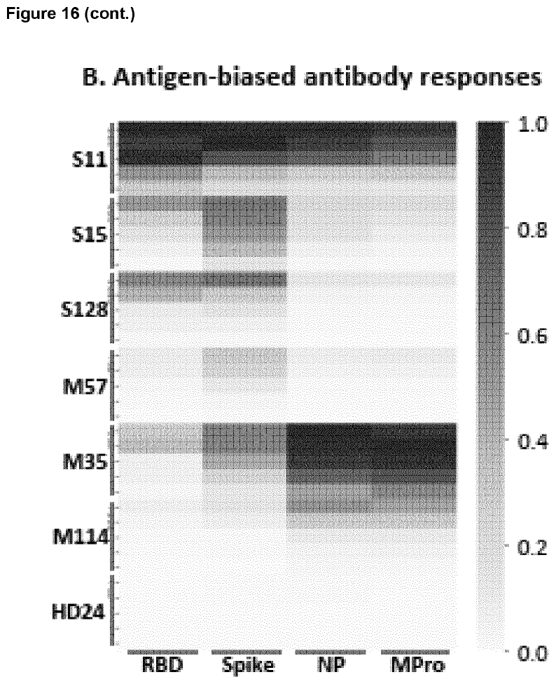

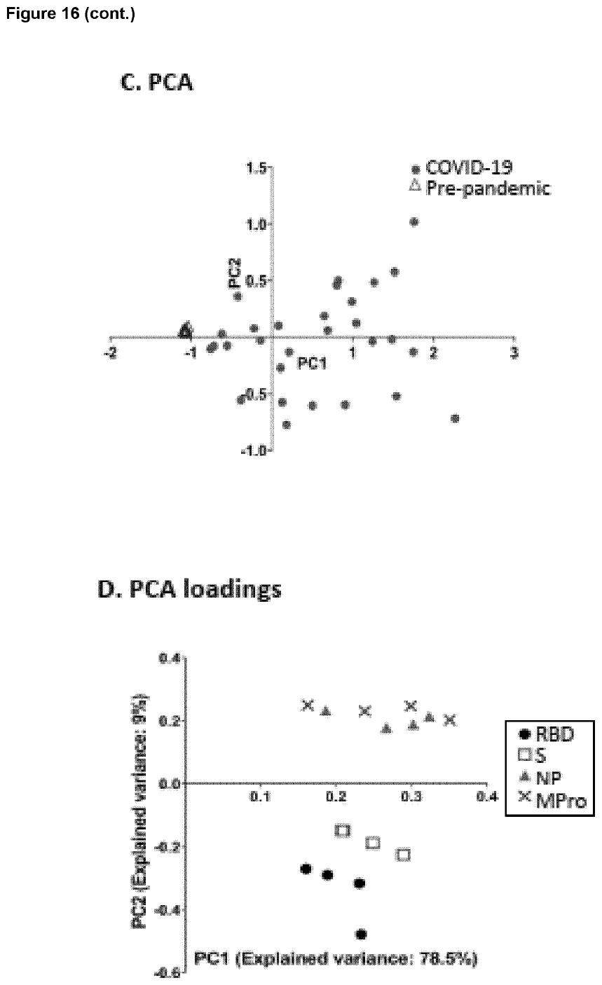

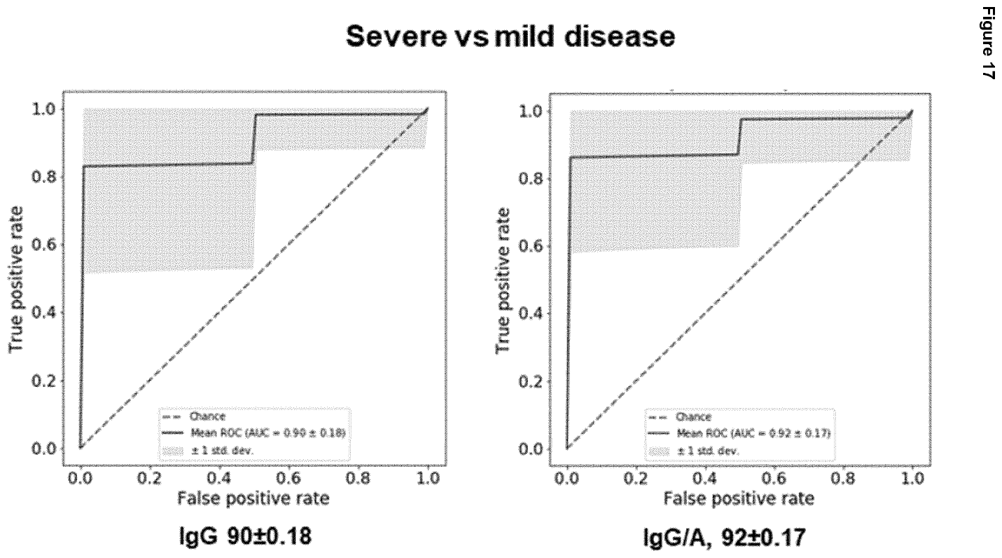

Using a small training set, ROC curves were generated to compare the sensitivity and specificity of each single-antigen ELISA test and for the multi-antigen FACS technique (FIG. 16A), and the latter again demonstrated the best performance, highlighting that a multi-antigen approach could be more useful in clinical contexts in which a high number of unknown samples must be classified using a limited amount of known controls.

The enhanced efficiency of the multi-antigen test is likely related to the observation that some patients clearly respond preferentially to antigens present in the viral particle (S, RBD), while other patients respond mainly to antigens normally only exposed once cells have been infected (NP, Mpro) (FIG. 16B). The existence of this bias was independently confirmed when a Principal Component Analysis (PCA) was performed with data for each antibody isotype. This analysis revealed a clear separation of seropositive and seronegative patients (FIG. 16C). Inspection of the PCA loadings (FIG. 16D) showed that, for IgG, the second principal component discriminated between production of antibodies against either NP+Mpro or S+RBD. Similar patterns were noted when IgA and IgM responses were analysed (not shown). The detection of this bias when analysing only a limited number of patients suggests that preferential antigen-specific responses are common and makes a strong case for the use of multi-antigen serological assays to avoid false-negative results. As the pandemic has advanced, it has been established that not all the patients respond in the same manner to the infection by SARS-CoV-2. In fact, a large body of clinical manifestations have been described and it has been suggested that different types of immune response may contribute to these different presentations. Therefore, it will likely be relevant to characterise potentially biased antibody responses when exploring the association between SARS CoV 2 infection and different clinical manifestations.

In aggregate, the multi-antigen assay produced data that easily and efficiently discriminated between seronegative and COVID seropositive individuals.

### Multi-Antigen, Multi-Isotype Analysis of COVID-19 Patients Antibody Response Improves Classification Related to Disease Severity and Allows Discrimination Between Vaccine-Induced and Naturally Infected Antibody Responses.

In general, patients with higher antibody titres are more likely to have suffered a severe infection, indicating infection severity is linked to increased antibody titres [13]. However, analysis of only IgG responses did not clearly discriminate between patients who had suffered severe or mild disease (FIG. 15). Statistically significant increased antibody responses in severe compared to mildly-affected patients were observed in the case of IgA antibodies against NP (dilutions 1:100-1:600), Mpro (dilutions 1:100-1:600) and RBD (dilutions 1:100-1:200). Using these variables to build a random forest allowed a classification into mild vs severe disease with a 92% accuracy (FIG. 17) when IgG data (dilutions 1:600-1800) and the IgA (dilution 1:100) responses were analysed simultaneously, compared to an accuracy of 90% when only IgG data is taken into account (FIG. 17). Although the analysis of a greater number of data is required, our results suggest the importance of analysing the IgG/IgA immune response against multiple SARS-CoV-2 antigens to establish clear criteria for severity discrimination.

1. An in vitro method for detecting in at least one biological sample an antibody that binds to at least one epitope of the SARS-CoV-2 virus, comprising:
a. contacting said at least one biological sample with at least one isolated SARS-CoV-2 M^{pro} protein, or at least one fragment of said isolated SARS-CoV-2 M^{pro} protein comprising at least one epitope of the SARS-CoV-2 virus, and
b. detecting the formation of an antigen-antibody complex between said virus protein or said fragment and an antibody present in said biological sample,
wherein said method is an in vitro diagnostic method for the detection of a subject having antibodies against the SARS-CoV-2 virus, wherein said subject is diagnosed as having antibodies against the SARS-CoV-2 virus if an antigen-antibody complex between said virus protein, or said fragment, and an antibody present in said biological sample is detected.

2. The in vitro method of claim 1, wherein said method is capable of detecting IgG, IgM and/or IgA.

3. The in vitro method of any of claim 1 or 2, wherein said method is capable of detecting IgG.

4. The in vitro method of any of claim 1 or 2, wherein said method is capable of detecting IgM.

5. The in vitro method of any of claim 1 or 2, wherein said method is capable of detecting IgA.

6. The in vitro method of any of claims 1 to 5, wherein said at least one isolated SARS-CoV-2 M^{pro} protein is the protein of SEQ ID NO 1 or a variant of SEQ ID NO 1 having at least 80% sequence identity to SEQ ID NO 1.

7. The in vitro method of any of claims 1 to 5, wherein said at least one fragment of said isolated SARS-CoV-2 M^{pro} protein comprising at least one epitope of the SARS-CoV-2 virus, comprises at least 20 contiguous amino acid residues having at least 80% sequence identity with at least about 20 contiguous amino acid residues of SEQ ID No. 1.

8. The in vitro method of any of claims 1 to 5, wherein said at least one isolated SARS-CoV-2 M^{pro} protein is the protein of SEQ ID NO 1.

9. The in vitro method of any of claims 1 to 8, wherein said at least one isolated SARS-CoV-2 M^{pro} protein or fragment thereof is a recombinant expression product.

10. The in vitro method of any of claims 1 to 9, wherein said biological sample is a blood, plasma or serum sample.

11. The in vitro method of claim 10, wherein said biological sample is a serum sample, and said SARS-CoV-2 M^{pro} protein is the protein of SEQ ID NO 1.

12. An in vitro kit suitable for detecting in a biological sample an antibody that binds to at least one epitope of the SARS-CoV-2 virus comprising:
a. at least one isolated SARS-CoV-2 M^{pro} protein, or at least one fragment of said isolated SARS-CoV-2 M^{pro} protein comprising at least one epitope of the SARS-CoV-2 virus, and
b. reagents for detecting the formation of antigen-antibody complex between said at least one isolated SARS-CoV-2 M^{pro} protein, or a fragment thereof, and at least one antibody present in a biological sample,
wherein said at least one isolated protein or fragment thereof and said reagents are present in an amount sufficient to detect the formation of said antigen-antibody complex.

13. The kit of claim 12, wherein said at least one isolated SARS-CoV-2 M^{pro} protein is the protein of SEQ ID NO 1 or a variant of SEQ ID NO 1 having at least 80% sequence identity to SEQ ID NO 1.

14. The kit of claim 12, wherein said at least one fragment of said isolated SARS-CoV-2 M^{pro} protein comprising at least one epitope of the SARS-CoV-2 virus, is characterized by comprising at least 20 contiguous amino acid residues having at least 80% sequence identity with at least 20 contiguous amino acid residues of SEQ ID No. 1.

15. The kit of any of claims 12 to 14, wherein said at least one isolated SARS-CoV-2 M^{pro} protein or fragment thereof is a recombinant expression product.

16. The kit of any of claims 12 to 15, wherein said at least one isolated SARS-CoV-2 M^{pro} protein is the protein of SEQ ID NO 1.

17. The kit of any of claims 12 to 16, wherein said kit is an ELISA system comprising at least one isolated SARS-CoV-2 M^{pro} protein of SEQ ID NO 1, to coat or coating a solid surface, preferably microtiter plate wells, and one or more of the following reagents: blocking reagents for unbound sites to prevent false positive results; anti-(species) IgG, IgM and/or IgA conjugated to a label, preferably an enzyme; and substrates that react with the label, preferably the enzyme, to indicate a positive reaction.

18. The in vitro method of any of claims 1 to 11, wherein the formation of antigen-antibody complex is detected by radioimmunoassay (RIA), enzyme linked immunosorbent assay (ELISA), chemiluminescent or colorimetric enzyme linked immunosorbent assay (ELISA), immunofluorescence assay (IFA), dot blot, a lateral flow immunochromatographic assay or western blot.

19. The in vitro method of any of claims 1 to 11, wherein the formation of antigen-antibody complex is detected by enzyme linked immunosorbent assay (ELISA).

20. The in vitro method of any of claims 1 to 11 or 18 and 19, wherein the formation of antigen-antibody complex is detected by any of the methods identified in claim 18 and said at least one virus protein or said fragment is adapted to detect IgG, IgM and/or IgA at a dilution of between 1:200 to 1:1800.

21. The in vitro method of any of claims 1 to 11 or 18 to 19, wherein the formation of antigen-antibody complex is detected by any of the methods identified in claim 18 and said is capable of detecting IgG, IgM and/or IgA at a dilution of between 1:200 to 1:1800.

22. The in vitro method of any of claims 1 to 11 or 18 to 21, wherein said biological sample is contacted with at least one or more further SARS-CoV-2 M^{pro} immunogens or fragments thereof.

23. The in vitro method of claim 22, wherein said further immunogens are selected from the group consisting of nucleocapsid (N) proteins of SARS-CoV-2, and spike (S) domains including the S1 subunit, and/or receptor binding domain (RBD) of SARS-CoV-2.

24. The in vitro method of any of claims 1 to 11 or 18 to 23, wherein said biological sample is contacted with at least one or more further immunogens derived from at least one distinct isolated SARS protein.

25. An ELISA system comprising at least one isolated SARS-CoV-2 M^{pro} protein or fragments thereof, as defined in any of claims 1 to 11, to coat or coating a solid surface, preferably microtiter plate wells, and one or more of the following reagents: blocking reagents for unbound sites to prevent false positive results; anti-(species) IgG, IgM and/or IgA conjugated to a label, preferably an enzyme; and substrates that react with the label, preferably the enzyme, to indicate a positive reaction.

26. The ELISA system of claim 25, wherein it further comprises additional reagents such as wash buffers, stop solutions and stabilizers.

27. In vitro use of the kit of any of claims 12 to 17 or the ELISA system of any of claim 25 or 26, for use in the implementation of the method of any of claims 1 to 11 or 18 to 24.


SEQUENCE LISTING (14 sequences)

SEQ ID NO: 1 - protein, 319 aa, Artificial Sequence
  PEPTIDE 1..319
  Peptide sequence SARS-CoV-2 Mpro
SAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQLEHHHHHH

SEQ ID NO: 2 - DNA, 960 bp, Artificial Sequence
  Nucleotide sequence of SARS-CoV-2 Mpro
  misc_feature 1..960
tcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaactcgagcaccaccaccaccaccactga

SEQ ID NO: 3 - protein, 377 aa, Artificial Sequence
  PEPTIDE 1..377
  Peptide sequence SARS-CoV-2 nucleocapsid protein
MASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPVEHHHHHH

SEQ ID NO: 4 - DNA, 1134 bp, Artificial Sequence
  Nucleotide sequence of SARS-CoV-2 nucleocapsid protein
  misc_feature 1..1134
atggcttctgataatggtccgcaaaatcagcgtaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctgtcgagcaccaccaccaccaccactga

SEQ ID NO: 5 - protein, 202 aa, Artificial Sequence
  PEPTIDE 1..202
  Peptide sequence SARS-CoV-2 RBD
GSRNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKLVPR

SEQ ID NO: 6 - DNA, 606 bp, Artificial Sequence
  Nucleotide sequence of SARS-CoV-2 RBD
  misc_feature 1..606
ggatcccgcaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaacttgtccctcgc

SEQ ID NO: 7 - DNA, 34 bp, Artificial Sequence
  Oligo
  misc_feature 1..34
gacccatggcttcagctgtttttcagagtggttt

SEQ ID NO: 8 - DNA, 32 bp, Artificial Sequence
  Oligo.
  misc_feature 1..32
gacctcgagttggaaagtaacacctgagcatt

SEQ ID NO: 9 - DNA, 44 bp, Artificial Sequence
  Oligo
  misc_feature 1..44
gatccatggcttctgataatggtccgcaaaatcagcgtaatgca

SEQ ID NO: 10 - DNA, 29 bp, Artificial Sequence
  Oligo
  misc_feature 1..29
caggtcgacaggctctgttggtgggaatg

SEQ ID NO: 11 - protein, 303 aa, Coronaviridae
  HCoVNL63
SGLKKMAQPSGCVERCVVRVCYGSTVLNGVWLGDTVTCPRHVIAPSTTVLIDYDHAYSTMRLHNFSVSHNGVFLGVVGVTMHGSVLRIKVSQSNVHTPKHVFKTLKPGDSFNILACYEGIASGVFGVNLRTNFTIKGSFINGACGSPGYNVRNDGTVEFCYLHQIELGSGAHVGSDFTGSVYGNFDDQPSLQVESANLMLSDNVVAFLYAALLNGCRWWLCSTRVNVDGFNEWAMANGYTSVSSVECYSILAAKTGVSVEQLLASIQHLHEGFGGKNILGYSSLCDEFTLAEVVKQMYGVNLQ

SEQ ID NO: 12 - protein, 302 aa, Coronaviridae
  HCoV229E
AGLRKMAQPSGFVEKCVVRVCYGNTVLNGLWLGDIVYCPRHVIASNTTSAIDYDHEYSIMRLHNFSIISGTAFLGVVGATMHGVTLKIKVSQTNMHTPRHSFRTLKSGEGFNILACYDGCAQGVFGVNMRTNWTIRGSFINGACGSPGYNLKNGEVEFVYMHQIELGSGSHVGSSFDGVMYGGFEDQPNLQVESANQMLTVNVVAFLYAAILNGCTWWLKGEKLFVEHYNEWAQANGFTAMNGEDAFSILAAKTGVCVERLLHAIQVLNNGFGGKQILGYSSLNDEFSINEVVKQMFGVNLQ

SEQ ID NO: 13 - protein, 305 aa, Coronaviridae
  SARS-CoV-2
GFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQ

SEQ ID NO: 14 - protein, 303 aa, Coronaviridae
  HCoVOC43
SGIVKMVNPTSKVEPCVVSVTYGNMTLNGLWLDDKVYCPRHVICSASDMTNPDYTNLLCRVTSSDFTVLFDRLSLTVMSYQMRGCMLVLTVTLQNSRTPKYTFGVVKPGETFTVLAAYNGKPQGAFHVTMRSSYTIKGSFLCGSCGSVGYVIMGDCVKFVYMHQLELSTGCHTGTDFNGDFYGPYKDAQVVQLPIQDYIQSVNFLAWLYAAILNNCNWFIQSDKCSVEDFNVWALSNGFSQVKSDLVIDALASMTGVSLETLLAAIKRLKNGFQGRQIMGSCSFEDELTPSDVYQQLAGIKLQ